(19) United States
(12) Patent Application Publication
(10) Pub. No.: US 2023/0035479 A1
(43) Pub. Date: Feb. 2, 2023

(54) METHODS AND COMPOSITIONS FOR REDUCING HAIR GREYING

(71) Applicants: President and Fellows of Harvard College, Cambridge, MA (US); The General Hospital Corporation, Boston, MA (US)

(72) Inventors: Ya-Chieh Hsu, Cambridge, MA (US); Bing Zhang, Cambridge, MA (US); David E. Fisher, Newton, MA (US); Inbal Rahamin, Brookline, MA (US)

(21) Appl. No.: 17/762,026

(22) PCT Filed: Mar. 25, 2020
(86) PCT No.: PCT/US20/24772

§ 371 Date: Mar. 18, 2022

Related U.S. Application Data
(60) Provisional application No. 62/964,613, filed on Jan. 22, 2020.
(60) Provisional application No. 62/903,517, filed on Sep. 20, 2019.

(51) Int. Cl. A61K 31/135; A61K 31/496; A61K 38/48; A61K 9/00; A61Q 5/00

(52) U.S. Cl. CPC A61K 31/135; A61K 31/496; A61K 38/4893; A61K 9/0053; A61Q 5/00

(57) ABSTRACT

Disclosed herein are methods and compositions for reducing and/or preventing hair greying in a subject.

### RELATED APPLICATIONS

This application claims the benefit of U.S. Provisional Application No. 62/903,517, filed on Sep. 20, 2019, and U.S. Provisional Application No. 62/964,613, filed on Jan. 22, 2020, the contents of which are hereby incorporated by reference in their entirety.

### GOVERNMENT SUPPORT

This invention was made with government support under AR070825 awarded by the National Institutes of Health. The government has certain rights in the invention.

### BACKGROUND OF THE INVENTION

Stress affects people of all ages, genders, and occupations, and is thought to be a risk factor for numerous diseases and disorders. Despite their profound impact, whether and how external stressors lead to tissue changes, and if stress-related changes occur at the level of somatic stem cells, is not well understood. Establishing the mechanisms of these stress-induced tissue changes is crucial to understand if and how psychological states shape stem cell behaviors and tissue functions. Such mechanistic insights will also identify genes and pathways that may be useful for reducing or reverting the undesirable effects of stress on stem cell function and tissue homeostasis.

### SUMMARY OF THE INVENTION

Disclosed herein are methods of reducing and/or treating hair greying in a subject. The methods may comprise inhibiting melanocyte stem cell (MeSC) hyper proliferation or suppressing nerve activity.

In some embodiments, wherein MeSC hyperproliferation or nerve activity is inhibited by administering a neurotoxin (e.g., 6-hydroxy dopamine or botulinum toxin).

In some embodiments, inhibition of MeSC hyper proliferation comprises inhibiting secretion of norepinephrine from activated sympathetic nerves. In some embodiments, sympathetic nerves are deactivated, e.g., by ablation. In some embodiments, secretion of norepinephrine is inhibited by administering to the subject an agent selected from the group consisting of: guanethidine, xylocholine, bretylium, debrisoquin, and botulinum toxin.

In some embodiments, inhibition of MeSC hyper proliferation comprises inhibiting an adrenergic receptor (e.g., β2 adrenergic receptors). β2 adrenergic receptor may be inhibited by administering to the subject a beta blocker selected from the group consisting of: propranolol, atenolol, metoprolol, acebutolol, nadolol, sotalol, bisoprolol, penbutolol, timolol, betaxolol, labetalol, pindolol, careolol, and exmolol.

In some embodiments, MeSC hyper proliferation is inhibited by administering to the subject a cyclin dependent kinase (CDK) inhibitor or a BRAF inhibitor. A CDK inhibitor may be selected from the group consisting of: palbociclib, ribociclib, letrozole, fulvestrant, AT7519, flavopiridol, and dinaciclib. A BRAF inhibitor may be vemurafenib or dabrafenib.

In some embodiments, MeSC hyper proliferation is inhibited by administering a CDK inhibitor and/or a beta blocker to the subject. In some embodiments, the CDK inhibitor and/or the beta blocker is formulated as a pharmaceutical composition. In some embodiments, the CDK inhibitor and/or the beta blocker is administered topically or orally.

Also disclosed herein are methods of reducing and/or preventing hair greying in a subject. The methods may comprise inhibiting secretion of norepinephrine by administering a first agent to the subject.

In some embodiments, secretion of norepinephrine is inhibited by deactivating activated sympathetic nerves, e.g., by ablation. In some embodiments, the first agent is a cyclin dependent kinase (CDK) inhibitor selected from the group consisting of: palbociclib, ribociclib, letrozole, fulvestrant, AT7519, and flavopiridol. In some embodiments, the first agent is formulated as a pharmaceutical composition. In some embodiments, the first agent is administered topically.

In some embodiments, the methods further comprise inhibiting norepinephrine receptors, e.g., β2 adrenergic receptors, by administering a second agent to the subject. In some embodiments, the second agent is a beta blocker selected from the group consisting of: propranolol, atenolol, metoprolol, acebutolol, nadolol, sotalol, bisoprolol, penbutolol, timolol, betaxolol, labetalol, pindolol, careolol, and exmolol. In some embodiments, the second agent is formulated as a pharmaceutical composition. In some embodiments, the second agent is administered topically.

Also disclosed herein are methods of reducing and/or preventing hair greying in a subject. The methods may comprise inhibiting norepinephrine receptors by administering an agent to the subject.

In some embodiments, the norepinephrine receptors are adrenergic receptors, e.g., β2 adrenergic receptors. In some embodiments, the agent is a beta blocker selected from the group consisting of: propranolol, atenolol, metoprolol, acebutolol, nadolol, sotalol, bisoprolol, penbutolol, timolol, betaxolol, labetalol, pindolol, careolol, and exmolol. In some embodiments, the agent is formulated as a pharmaceutical composition. In some embodiments, the agent is administered topically.

Disclosed herein are pharmaceutical compositions comprising a first agent that inhibits MeSC proliferation.

Also disclosed herein are methods of causing or accelerating hair greying in a subject. The methods may comprise increasing levels of norepinephrine in the subject by administering an agent. The agent may be selected from norepinephrine, an Akt activator (e.g., FGF2 or SC-79), and an adrenergic beta agonist.

### BRIEF DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E demonstrate stress depletes melanocyte stem cells (MeSCs). FIG. 1A shows black coat C57BL/6J mice are subjected to different stress models. FIG. 1B shows hair greying after resiniferatoxin (RTX) injection. Right, quantification of skin area covered by white hairs (n=10 mice for each condition, two-tailed unpaired t-test). FIG. 1C shows liquid chromatography with tandem mass spectrometry (LC-MS-MS) quantifies serum stress hormone concentrations after injection of RTX alone or in combination with buprenorphine (Bup, n=6 mice for each condition, one-way ANOVA with Tukey's multiple comparisons). FIG. 1D shows injection of RTX with buprenorphine blocks white hair formation (n=6 mice for each condition, two-tailed unpaired t-test). FIG. 1E, upper panel, provides experimental design (black arrow: RTX injection, red arrows: harvesting). FIG. 1E, lower panels, shows immunofluorescent staining for TRP2 (melanocyte lineage marker) in the hair follicle (HF) of control (Ctrl, saline-injected) and RTX injected mice (n=30 HFs throughout the skin from 6 mice for each condition, two-way ANOVA with original FDR method of Benjamini and Hochberg). Yellow boxes denote the upper HF region where MeSCs reside. Enlarged views are shown to the right. Arrowheads: MeSCs. CUS: chronic unpredictable stress. D: day. Ana: anagen. Cata: catagen. Telo: telogen. Diff Mc: differentiated melanocytes. Scale bars, 50 μm. All data are mean ±S.D.

FIGS. 2A-2D demonstrate norepinephrine drives hair greying. FIG. 2A provides possible mechanisms of MeSC loss. FIG. 2B shows RTX injection into Tyr-CreER; adrb2 f1/f1 (MeSC-adrb2 cKO) mice fails to trigger hair greying (n=6 mice for each condition, two-tailed unpaired t-test). FIG. 2C shows white hair formation in norepinephrine injection sites (NE; n=10 injected sites from 8 mice for each condition. Quantifications see FIG. 9A). Yellow dashed circles denote intradermal injection sites. FIG. 2D shows white hair formation after RTX injection in adrenalectomized mice (ADX, n=6 mice for each condition, two-tailed unpaired t-test). All data are mean ±S.D.

FIGS. 3A-3E demonstrate hyperactivation of sympathetic nervous system drives MeSC. FIG. 3A shows sympathetic nerve innervates MeSC niches. White arrowhead indicates the close proximity of nerve endings to MeSCs (n=6 mice for each condition). FIG. 3B shows immunofluorescent staining of sympathetic ganglia for tyrosine hydroxylase (TH, green) and c-FOS (red) from mice injected with saline, RTX, and RTX with buprenorphine (n=6 ganglia from 3 mice for each condition, one-way ANOVA with Tukey's multiple comparisons). FIG. 3C shows 6-hydroxydopamine (6-OHDA) injection blocks MeSC loss and white hair induction by RTX (n=30 HFs from 6 mice for each condition, two-tailed unpaired t-test. See also FIG. 10D). FIG. 3D, left, provides a schematic of sympathetic nerve activation using a Gq-DREADD system. FIG. 3D, right, shows immunofluorescent staining for TH (green) and TRP2 (red) from TH-CreER; Gq-DREADD mice treated with saline or Clozapine N-Oxide (CNO, n=30 HFs from 6 mice for each condition, two-tailed unpaired t-test). FIG. 3E shows mosaic activation of sympathetic nerves using TH-CreER; Gq-DREADD; Rosa-mT/mG mice. Bar graphs quantify the number of MeSCs in HFs innervated by DREADD negative sympathetic nerves (w/o DREADD) vs. DREADD positive sympathetic nerves (w/DREADD, marked by membrane GFP expression). n=30 HFs for each condition from 4 mice, two-tailed unpaired t-test. SN abla: sympathetic nerve ablation. Scale bars, 50 μm. All data are mean ±S.D.

FIGS. 4A-4E demonstrate norepinephrine drives MeSCs out of quiescence. FIG. 4A provides possible mechanisms by which norepinephrine depletes MeSCs. FIG. 4B shows immunofluorescent staining for Phospho-Histone H3 (pHH3, green) and TRP2 (red) 1 day after RTX or norepinephrine injection. White arrowhead highlights the proliferative MeSCs (n=30 HFs from 5 mice for each condition, one-way ANOVA with Tukey's multiple comparisons). FIG. 1C shows time-course of MeSC behavior after RTX treatment in Tyr-CreER; R26-mT/mG mice. White arrowheads mark MeSCs (n=30 HFs from 3 mice for each timepoint, one-way ANOVA with Tukey's multiple comparisons). FIG. 4D shows Fontana—Masson melanin staining 5 days after saline or RTX injection (n=6 mice for each condition). Blue arrowheads indicate ectopic pigments. FIG. 4E provides a model summarizing steps of stress-induced MeSC depletion. TAM: tamoxifen. Scale bars, 50 μm. All data are mean ±S.D.

FIGS. 5A-5G demonstrate inhibition of aberrant MeSC proliferation prevents stress-induced hair greying. FIG. 5A provides experimental workflow. FACS at telogen. FIG. 5B provides a gene ontology enrichment analysis of significantly dysregulated genes in stressed MeSCs (n=2 biologically independent samples for each condition, Fisher exact test). FIG. 5C provide heatmaps of signature gene expression related to MeSC proliferation (n=2 biologically independent samples for each condition). FIG. 5D shows qRT-PCR of MeSC proliferation and differentiation genes in cultured primary human melanocytes treated with norepinephrine (n=6 samples from three independent donors, two-way ANOVA with original FDR method of Benjamini and Hochberg). FIG. 5E shows immunofluorescent staining for TRP2 (red) from mice 5 days after treatments of RTX together with AT7519, Flavopiridol, or with MeSC-specific P27 overexpression (P27 OE, n=30 HFs from 6 mice each condition, one-way ANOVA with Tukey's multiple comparisons). FIG. 5F shows topical treatment of AT7519, Flavopiridol, or MeSC-specific P27 overexpression inhibits RTX-induced hair greying (n=6 mice for each condition, one-way ANOVA with Tukey's multiple comparisons). FIG. 5G provides a model summarizing the main findings. Under strong external stressors, activated sympathetic nerves secrete norepinephrine that binds to ADRB2 on MeSCs. NE-ADRB2 signalling drives rapid MeSC proliferation, followed by ectopic differentiation and exhaustion. Flavo: Flavopiridol. Scale bars, 50 μm. All data are mean ±S.D.

FIGS. 6A-6D demonstrate effects of stress on the hair pigmentation. FIG. 6A provides a schematic of MeSCs behavior during hair cycle. FIG. 6B shows hair greying after mice are subjected to chronic unpredictable stress (CUS). Quantifications are done by plucking—100 hairs from different regions across the skin and counting the number of white hairs (n=9 plucked regions from 3 mice for each condition, two-tailed unpaired t-test). FIG. 6C shows hair greying after mice are subjected to restraint stress. Quantifications are done as described in FIG. 6B. FIG. 6D shows LC-MS-MS quantification of corticosterone and norepinephrine after restraint stress (n=5 mice for control and n=6 mice for restraint, two-tailed unpaired t-test). FIG. 6E shows immunofluorescent staining of hair bulbs for Melanocyte Inducing Transcription Factor (MITF, red) from mice 5 days after treatment of saline or RTX (n=30 HFs from 3 mice for each condition, two-tailed unpaired t-test). FIG. 6F shows Fontana—Masson staining of hair bulbs for melanin from mice 5 days after treatment of saline or RTX (n=6 mice for each condition). FIG. 6G shows hair coat color in mice 5 days after RTX injection in anagen. RTX is injected in full anagen and the mice are examined 5 days later at late anagen. The coat color remains black (n=6 mice for each condition). FIG. 6H shows Fontana—Masson staining of HFs for melanin from mice treated with saline or RTX at first anagen and examined at second anagen (see FIG. 1E, 2nd Ana for corresponding fluorescent images, n=6 mice for each condition). FIG. 61 provides quantification of MeSC numbers in saline and RTX-injected skins. For the RTX-injected skins, the number of MeSCs in regions with predominantly black hairs and regions with many white hairs are quantified separately. Orange and green dashed boxes denote representative black and white hair regions in RTX injected mice. Enlarged boxes contain representative immunofluorescent images of HFs from each region. White arrowheads indicate regions where MeSC reside. n=30 HFs from 3 mice for each condition, one-way ANOVA with Tukey's multiple comparisons. FIG. 6J provides quantification of the body area covered by white hairs in female vs. male mice (n=5 mice for each sex, two-tailed unpaired t-test). All data are mean ±S.D.

FIGS. 7A-7D demonstrate loss of MeSCs after three different stress models. FIG. 7A, upper panel, provides a schematic of experimental design for RTX injection in first telogen (red arrows indicate harvesting). FIG. 7A, lower panel left, provides representative mouse images 5 days and 16 days after RTX injection in first telogen. FIG. 7A, lower panel right, provides quantification of the body area covered by white hairs 16 days after RTX injection (n=4 mice for each condition, two-tailed unpaired t-test). FIG. 7B shows immunofluorescent staining for TRP2 from saline or RTX-injected mice (n=30 HFs from 4 mice for each condition, two-tailed unpaired t-test). Yellow boxes denote the upper HF region where MeSCs reside. Enlarged view of the yellow box regions are shown to the right. Arrowheads indicate MeSCs. FIG. 7C shows immunofluorescent staining for TRP2 (red) from mice subjected to CUS or restraint stress (n=30 HFs from 5 mice for each condition, two-tailed unpaired t-test). FIG. 7D shows hair coat color is monitored in RTX-injected mice for multiple rounds of hair follicle regeneration (waxing is used to initiate new rounds of anagen, n=3 mice for each condition). Schematic denotes the experimental design. Scale bars, 50 μm. All data are mean ±S.D.

FIGS. 8A-8E demonstrate stress-induced hair greying is not mediated through corticosterone or immune attack. FIG. 8A, left, shows white hair formation after RTX injection in Rag1 mutant mice devoid of T and B cells (Rag1 KO, n=6 for each condition, two-tailed unpaired t-test). FIG. 8A, right, shows immunofluorescent staining for T cell marker CD3 (green) in control and Rag1 KO skin (n=6 mice for each condition, two-tailed unpaired t-test). FIG. 8B, left, shows hair greying occurs when RTX is injected into CD11b-DTR mice treated with diphtheria toxin (DT) to deplete myeloid cells (n=6 mice for each condition). FIG. 8B, right, shows immunofluorescent staining for CD11b (green) in DT treated control and CD11b-DTR skin (n=6 mice for each condition). FIG. 8C shows expression of adrenergic receptors and glucocorticoid receptor (GR) in MeSCs (n=2 biologically independent samples). FIG. 8D shows white hair formation following RTX injection into Tyr-CreER; GR f1/f1 mice (MeSC-GR cKO; n=6 mice for each condition, two-tailed unpaired t-test). FIG. 8E, left, provides enzyme-linked immunosorbent assay (ELISA) measurement of corticosterone level in the blood 3 days after supplying corticosterone in drinking water (n=4 mice for each condition). FIG. 8E, middle, shows immunofluorescent staining of hair follicles for TRP2 (red) from mice 5 days after corticosterone treatment (n=30 HFs from 3 mice for each condition, two-tailed unpaired t-test). FIG. 8E, right, shows hair coat color after HFs in corticosterone-treated mice enter another round of anagen to regenerate new hairs. CORT: corticosterone. Scale bars, 50 μm. All data are mean ±S.D.

FIGS. 9A-9G demonstrate perturbations of the norepinephrine-ADRB2 pathway. FIG. 9A shows immunofluorescent staining of HFs for Phospho-CREB (green) and TRP2 (red) 12 hours after RTX injection (n=30 HFs from 3 mice for each condition, two-tailed unpaired t-test). White arrowheads indicate Phospho-CREB positive MeSCs in upper HFs after RTX injection. FIG. 9B shows white hair formation following RTX injection into K15-CrePGR; adrb2 f1/f1 mice (HFSC-Adrb2 cKO; n=3 mice for each condition, two-tailed unpaired t-test). FIG. 9C, upper left, shows hair coat color in unstressed Tyr-CreER; adrb2 f1/f1 mice (MeSC-adrb2 cKO) in the second telogen after 7× tamoxifen treatment at the first telogen. FIG. 9C, lower left, shows immunofluorescent staining of hair bulbs for MITF (red) in Tyr-CreER; adrb2 f1/f1 mice in anagen. FIG. 9C, right, shows Fontana—Masson melanin staining of anagen HFs from Tyr-CreER; adrb2 f1/f1 mice (n=3 mice for each condition). FIG. 9D, upper left, provides a schematic of experimental design for mosaic labelling in unstressed control and adrb2 knockout (red arrows indicate harvesting). FIG. 9D, lower left, shows immunofluorescent staining for GFP (green) and TRP2 (red) from Tyr-CreER; R26-mT/mG mice (MeSC-mT/mG) and Tyr-CreER; adrb2 R26-mT/mG mice (MeSC-adrb2 cKO-mT/mG) after 3× tamoxifen treatment at first telogen. FIG. 9D, right, shows immunofluorescent staining of HFs for GFP (green) and TRP2 (red) after the mice enter anagen (n=3 mice for each condition, TAM: tamoxifen). FIG. 9E provides quantification of white hair percentage after intradermal injection of saline or norepinephrine (n=10 injected sites from 6-8 mice for each condition, one-way ANOVA with Tukey's multiple comparisons). FIG. 9F shows immunofluorescent staining of HFs for TRP2 (red) from mouse skins intradermally injected with NE (n=30 HFs from 10 injection sites for each condition, one-way ANOVA with Tukey's multiple comparisons). FIG. 9G shows white hairs are formed after intradermal injection of NE in K15-CrePGR, adrb2 f1/f1 mice (HF-adrb2 cKO, n=3 injection sites for each condition, two-tailed unpaired t-test). Yellow dashed circles denote intradermal injection sites. Scale bars, 50 μm. All data are mean ±S.D.

FIGS. 10A-10H demonstrates activation of the sympathetic nervous system by nociception-induced stress or sympathetic nerve-specific inducible Gq-DREADD. FIG. 10A provides LC-MS/MS quantification of stress hormones in sham-operated and adrenalectomized mice (ADX, n=3 mice for each condition, two-way ANOVA with original FDR method of Benjamini and Hochberg). FIG. 10B, upper panel, shows immunofluorescent staining of sympathetic nerves in the skin regions with predominantly black hairs (orange box) and regions with mostly white hairs (green box, n=3 mice for each condition). FIG. 10B, lower panel, shows 3D surfaces of tyrosine hydroxylase (TH) staining created using Imaris software and quantification of sympathetic nerve volume from regions with different number of unpigmented hairs (n=20 hair follicles (HFs) for each region from 3 mice, two-tailed unpaired t-test). FIG. 10C shows immunofluorescent staining of sympathetic ganglia for TH (green) and c-FOS (red) from mice injected with RTX and harvested at different time points between 0 to 24 hours (n=6 sympathetic ganglia from 3 mice for each time points). FIG. 10D provides quantification of chemical sympathectomy efficiency (n=6 mice for each condition, two-tailed unpaired t-test) and % of white hairs in RTX-injected mice treated with vehicle or 6-OHDA (n=6 mice for each condition, two-tailed unpaired t-test). FIG. 10E shows Guanethidine (Gua) injection blocks formation of white hairs induced by RTX injection (quantification for % of white hairs: n=14 mice for each condition, two-tailed unpaired t-test; quantification for MeSC numbers: n=30 HFs from 6 mice for each condition, two-tailed unpaired t-test). FIG. 10F shows immunofluorescent staining of sympathetic ganglia for TH (green) and c-FOS (red) from TH-CreER; Gq-DREADD mice injected with CNO and harvested 6 hours later (n=6 sympathetic ganglia from 2 mice for each condition, two-tailed unpaired t-test). FIG. 10G shows white hair formation after intradermal injection of CNO into TH-CreER, Gq-DREADD mice (n=6 injection sites from 5 mice for each condition, two-tailed unpaired t-test). Yellow dashed circles denote intradermal CNO injection sites. FIG. 10H provides quantification of white hair percentage on CNO injection sites in mosaically-induced TH-CreER; Gq-DREADD; R26-mT/mG mice (n=5 injection sites from 4 mice for each condition, two-tailed unpaired t-test). Scale bars, 50 μm. All data are mean ±S.D.

FIGS. 11A-11I demonstrate apoptosis and proliferation analysis of MeSCs and the impact of RTX or norepinephrine on mature melanocytes. FIG. 11A shows immunofluorescent staining of active Caspase3 (aCAS3, green) and TRP2 (red) from mice 1 day after RTX or NE injection (n=30 HFs from 6 mice for each condition, one-way ANOVA with Tukey's multiple comparisons). FIG. 11B shows terminal deoxynucleotidyl transferase dUTP nick end labelling (TUNEL) assay of HFs from mice 1 day after RTX or NE treatment. Catagen HFs are used as positive controls for TUNEL. White arrowhead points to apoptotic hair follicle cells (n=30 HFs from 6 mice for each condition, one-way ANOVA with Tukey's multiple comparisons). FIG. 11C shows white hair formation in RIPK3 mutant mice (RIPK3 KO) injected with RTX (n=5 mice for each condition, two-tailed unpaired t-test). FIG. 11D shows immunofluorescent staining of HFs for the DNA damage marker y-H2AX (green) and TRP2 (red) from mice 1 day after RTX or NE treatment. HFs from irradiated mice are used as positive controls. White arrowhead indicates the MeSCs with DNA damage (n=30 HFs from 6 mice for each condition, one-way ANOVA with Tukey's multiple comparisons). FIG. 11E shows immunofluorescent staining for pHH3 (green) and TRP2 (red) of control HFs at different hair cycle stages (n=25 HFs from 3 mice for each condition, one-way ANOVA with Tukey's multiple comparisons). FIG. 11F shows immunofluorescent staining of hair bulbs for aCAS3 (green) and TRP2 (red) from mice 1 day after RTX or NE injection (n=30 HFs from 3 mice for each condition, one-way ANOVA with Tukey's multiple comparisons). FIG. 11G shows immunofluorescent staining of hair bulbs for pHH3 (green) and TRP2 (red) from mice 1 day after RTX or NE injection (n=30 HFs from 3 mice for each condition, one-way ANOVA with Tukey's multiple comparisons). FIG. 11H, left panel, provides a schematic of MeSCs isolation strategy. FIG. 11H, right panel, provides FACS analysis of MeSC numbers 1 day after RTX (n=5 mice for each condition, two-tailed unpaired t-test). FIG. 11I shows Fontana—Masson melanin staining of anagen or telogen samples 5 days after saline or RTX injection (n=6 mice for each condition, two-tailed unpaired t-test). Blue arrowheads indicate ectopic pigments. Scale bars, 50 μm. All bar graphs are mean ±S.D.

FIGS. 12A-12G demonstrate differential gene expression in normal and stressed MeSCs. FIG. 12A shows FACS strategy for MeSCs purification. MeSCs are selected based on their expression of CD117, from a population that is negative for CD140a, CD45, Sca1, CD34, and modest expression for Integrin alpha-6. FIG. 12B shows sample clustering based on Pearson's correlation of transcriptome among control and stressed MeSCs (n=2 biologically independent samples for each condition). FIG. 12C provides a heatmap of all differentially expressed genes (n=2 biologically independent samples for each condition, P values calculated using Wald test implemented in DESeq2, and adjusted using the Benjamini—Hochberg method. Log2FoldChange ≥0.58 and adjusted p value <0.05). FIG. 12D shows expression level of marker genes for different cell types in the skin confirming the purity of MeSCs used for RNA-seq (n=4 biologically independent samples). FIG. 12E provides heatmaps showing expression of signature genes related to MeSC differentiation. FIG. 12F provides heatmaps illustrating expression of cell cycle signature genes. FIG. 12G shows qRT-PCR validation of selected differentially expressed genes in FACS-purified mouse MeSCs from control and RTX injected skins (n=4 biological replicates for each condition, two-way ANOVA with original FDR method of Benjamini and Hochberg). All data are mean ±S.D.

FIGS. 13A-13B provide proliferation analysis of RTX-injected mice treated with CDK inhibitors chemically or genetically. FIGS. 13A-13B shows immunofluorescent staining of upper HFs and hair bulbs for pHH3 (green) and TRP2 (red) from mice 1 day after RTX injection together with topical application of CDK inhibitors (AT7519 or Flavopiridol) or with MeSC-specific P27 overexpression (MeSC-P27 OE, n=30 HFs from 3 mice for each condition, one-way ANOVA with Tukey's multiple comparisons). Scale bars, 50 μm. All data are mean ±S.D.

FIGS. 14A-14D demonstrate that norepinephrine stimulates MeSC differentiation in human hair follicles. FIG. 14A shows norepinephrine induces MITF and multiple MITF target genes in cultured primary human melanocytes. FIG. 14B shows norepinephrine (10 uM) induces transient phosphorylation/activation of CREB/ATF1 in human primary melanocytes after 15 minutes, indicating the presence of a functional receptor and induction of cAMP signaling. FIG. 14C provides quantification of the proportion of follicles that contain pigmented bulge or outer root sheath cells vs those lacking such pigmented cells outside of the bulb region. Human discarded hair bearing skin was cultured for 3 days with single hairs being isolated and grown in semi solid agarose William E medium with vehicle or norepinephrine (0.1 mM) for 24 hours. The cells were assessed after culture to identify those that were pigmented from those that were not. The presence of pigmented cells indicates senescence of melanocyte stem cells. Signal in vehicle control likely reflects the middle-age of the donor follicles. FIG. 14D provides an example of heavy norepinephrine-induced melanocyte stem cell pigmentation (compare left vs middle panels) Immunofluorescence staining was carried out for the pigment enzyme DCT (red cytoplasmic stain) to confirm that the pigmented cells were indeed melanocytes (right panel).

FIG. 15 shows beta-blockade prevents norepinephrine-induced melanocyte pigmentation/senescence in human hair follicles. Hair follicles from discarded scalp skin of 45 a year old male was cultured for 3 days. Single hairs were isolated and grown in a semi agarose William E medium with vehicle, norepinephrine (NE) (0.1 mM), or NE (0.1 mM) +butoxamine (0.1 mM) (β2 blocker) for 24 hours. The graph indicates the proportion of follicles containing (or lacking) pigmented melanocytes (see arrows) in the bulge or outer root sheath locations, indicating melanocyte stem cell differentiation/senescence.

### DETAILED DESCRIPTION OF THE INVENTION

Hair greying was found to result from activation of sympathetic nerves that innervate the MeSC niche. Sympathetic nerve activation leads to burst release of the neurotransmitter norepinephrine (also known as noradrenaline), which drives quiescent MeSCs into rapid proliferation, followed by differentiation, migration, and permanent depletion from the niche. Transient suppression of MeSC proliferation prevents hair greying.

Sympathetic nerve activation may occur as a result of stress. Combining adrenalectomy, denervation, chemogenetics [3,4], cell ablation, and MeSC-specific adrenergic receptor knockout, it was demonstrated that stress-induced MeSC loss is independent of immune attack or adrenal stress hormones. In some cases, sympathetic nerve activity is also known to be elevated with age. Moreover, sympathetic nerve is active at a basal level. It is demonstrated herein that acute stress-induced neuronal activity can drive rapid and permanent loss of MeSCs. The basal level of sympathetic nerve activity may gradually deplete the MeSC population as well.

Methods of Treatment

The disclosure of the invention is directed to methods of delaying, inhibiting, reducing and/or treating hair greying in a subject. Hair greying (e.g., stress-induced hair greying) may be reduced in a subject by inhibiting or suppressing melanocyte stem cell (MeSC) hyper proliferation. In some embodiments, MeSC hyper or aberrant proliferation is inhibited by inhibiting the sympathetic nervous system.

The sympathetic nervous system may become activated in response to stress. Activation of the sympathetic nervous system results in the release or secretion of norepinephrine, for example, from peripheral axon terminals. Sympathetic nerves terminate close to the bulge where MeSCs reside and MeSCs include the β2 adrenergic receptor (ADRB2), a receptor for norepinephrine. The release of norepinephrine from the activated sympathetic nerves may signal through ADRB2 on MeSCs.

In some embodiments, hair greying (e.g., stress induced hair greying) in a subject is treated or prevented by administration (e.g., topical or oral administration) of one or more agents. In some embodiments, MeSC hyper proliferation is inhibited by administration of one or more agents. Exemplary types of agents that can be used include small organic or inorganic molecules; saccharines; oligosaccharides; polysaccharides; a biological macromolecule selected from the group consisting of peptides, proteins, peptide analogs and derivatives; peptidomimetics; nucleic acids selected from the group consisting of siRNAs, shRNAs, antisense RNAs, ribozymes, and aptamers; an extract made from biological materials selected from the group consisting of bacteria, plants, fungi, animal cells, and animal tissues; naturally occurring or synthetic compositions; antibodies; and any combination thereof. In some embodiments, MeSC hyper proliferation is inhibited using any gene editing tool known to those of skill in the art (e.g., TALENS, CRISPR, ZFN, etc.).

In some embodiments, MeSC hyper proliferation is inhibited or suppressed by inhibiting the activation of sympathetic nerves. For example, sympathetic nerves may be ablated by applying or administering an agent. In some embodiments, an agent for ablating sympathetic nerves is a selective neurotoxin. In some aspects, sympathetic nerves are ablated with a selective neurotoxin for sympathetic nerves. In some embodiments, the selective neurotoxin is 6-hydroxy dopamine (6-OHDA) or botulinum toxin (botox). In some embodiments, MeSC hyper proliferation is inhibited by administering an agent for inhibiting sympathetic nerves. In some embodiments, MeSC hyperproliferation is inhibited by administering a sympathetic nerve neurotoxin, e.g., 6-OHDA or botox. Hair greying in a subject may be treated or prevented by administration of a sympathetic nerve neurotoxin, e.g., 6-OHDA or botox.

In some embodiments, MeSC hyper proliferation is inhibited or suppressed by inhibiting the release of norepinephrine by sympathetic nerves. The release or secretion of norepinephrine, e.g., from sympathetic nerve terminals, may be inhibited or blocked by administering an agent. Non-limiting examples of an agent for blocking the release of norepinephrine include guanethidine, xylocholine, bretylium, debrisoquin, and botulinum toxin. In some embodiments, MeSC hyperproliferation is inhibited by administering an agent for inhibiting the release of norepinephrine. In some embodiments, MeSC hyperproliferation is inhibited by administering guanethidine. Hair greying in a subject may be treated or prevented by administration of an agent for inhibiting the release of norepinephrine from sympathetic nerves, e.g., guanethidine.

In some embodiments, MeSC hyper proliferation is inhibited or suppressed by blocking a norepinephrine receptor, e.g., an adrenergic receptor. In some embodiments, the adrenergic receptor is the β2 adrenergic receptor. The adrenergic receptor may be blocked or inhibited by administration of an agent. For example, the adrenergic receptor may be blocked by administration of a beta blocker (e.g., a non-selective beta blocker or a selective beta blocker). In some aspects, a beta blocker contains or possesses an activity that blocks β2 adrenergic receptor. In some embodiments, adrenergic receptor blockers include, but are not limited to, propranolol, atenolol, metoprolol, acebutolol, nadolol, sotalol, bisoprolol, penbutolol, timolol, betaxolol, labetalol, pindolol, careolol, and exmolol. In some embodiments, MeSC hyperproliferation is inhibited by administering an agent for blocking the β2 adrenergic receptor. In some embodiments, MeSC hyperproliferation is inhibited by administering a beta blocker. Hair greying in a subject may be treated or prevented by administration of an agent for blocking the β2 adrenergic receptor, e.g., a beta blocker.

In some embodiments, MeSC hyper proliferation is inhibited or suppressed by administering an agent that transiently suppresses proliferation. For example, MeSC hyper proliferation may be inhibited by administering a cell cycle inhibitor, e.g., a cyclin-dependent kinase (CDK) inhibitor and/or by administering a BRAF inhibitor. In some aspects a CDK inhibitor is an inhibitor of CDK2, CDK4, and/or CDK6. Non-limiting examples of CDK inhibitors include alsterpaullone, aminopurvalanol A, arcyriaflavin A, AZD 5438, BIO, BMS 265246, BRD 6989, BS 181 dihydrochyloride, CGP 60474, CGP 74514 dihydrochloride, (R)-CR8, CVT 313, (R)-DRF053 dihydrochloride, flavopiridol, indirubin-3′-oxime, kenpaullone, LDC 000067, NSC 625987, NSC 663284, NSC 693868, NU 2058, NU 6140, NVP 2, olomoucine, [Ala⁹²]-p16 (84-103), palbociclib, PHA 767491 hydrochloride, purvalanol A, purvalanol B, R 547, ribociclib, Ro3306, roscovitine, ryuvidine, senexin A, SNS 032, SU 9516, letrozole, fulvestrant, dinaciclib, and AT7519. Non-limiting examples of BRAF inhibitors include vemurafenib and dabrafenib. In some aspects, MeSC hyper proliferation is inhibited by inducing expression of a CDK inhibitor, e.g., P27, P21, or P57. Expression of a CDK inhibitor may be induced by administration of doxycycline. In some embodiments, MeSC hyper proliferation is inhibited by administering a CDK inhibitor. Hair greying in a subject may be treated or prevented by administration of an agent for inhibiting MeSC hyper proliferation, e.g., a CDK inhibitor.

In some embodiments, methods of reducing and/or treating hair greying (e.g., stress induced hair greying) in a subject comprise administering one or more of an agent(s) for inhibiting release of norepinephrine from sympathetic nerves, an agent(s) for blocking the β2 adrenergic receptor, and an agent(s) for inhibiting proliferation of MeSC.

Also disclosed herein are methods of reducing and/or treating hair greying (e.g., stress-induced hair greying or hair greying in general) in a subject comprising inhibiting secretion of norepinephrine from sympathetic nerves (e.g., activated sympathetic nerves). In some embodiments, secretion of norepinephrine is inhibited by the administration of an agent to the subject. Also disclosed herein are methods of reducing and/or treating hair greying (e.g., stress-induced hair greying) in a subject comprising blocking β2 adrenergic receptors. In some embodiments, β2 adrenergic receptors are blocked by administration of an agent to the subject. Also disclosed herein are methods of reducing and/or treating hair greying (e.g., stress-induced hair greying) in a subject comprising inhibiting secretion of norepinephrine and blocking β2 adrenergic receptors (e.g., by administration of a first and a second agent).

Also disclosed herein are methods of causing and/or increasing hair greying in a subject comprising increasing proliferation of MeSC. In some embodiments, an agent is administered to the subject to increase proliferation of MeSC. In some embodiments, proliferation of MeSCs is driven by elevating levels of norepinephrine. In some embodiments, proliferation of MeSCs is increased by administering norepinephrine to the subject. In some embodiments, proliferation of MeSCs is increased by administering an Akt activator (e.g., FGF2 or SC-79). In some embodiments, proliferation of MeSCs is increased by administering a beta agonist (e.g., an adrenergic beta agonist). Non-limiting examples of adrenergic beta agonists are described at drugbank.ca/categories/DBCAT000553, incorporated herein by reference. In some aspects, proliferation of MeSCs is increased by administering an agent or construct via Clozapine N-Oxide (CNO) to activate sympathetic nerves. Methods of causing and/or increasing hair greying in a subject may comprise increasing levels of norepinephrine in the subject. For example, a subject may be administered norepinephrine or an agent that increases secretion of norepinephrine. In some aspects, methods of causing and/or increasing hair greying in a subject may comprise activating the sympathetic nervous system in the subject. For example, a subject may be administered an agent that activates sympathetic nerves (e.g., CNO).

Agents and Pharmaceutical Compositions

The disclosure contemplates agents that inhibit and/or treat hair greying (e.g., stress-induced hair greying) in a subject. In some aspects, agents inhibit MeSC proliferation (e.g., hyper or aberrant proliferation). In some embodiments, an agent that inhibits MeSC proliferation is a cell cycle inhibitor, e.g., a cyclin-dependent kinase (CDK) inhibitor or a BRAF inhibitor. In some embodiments, an agent is a CDK inhibitor selected from the group consisting of: alsterpaullone, aminopurvalanol A, arcyriaflavin A, AZD 5438, BIO, BMS 265246, BRD 6989, BS 181 dihydrochyloride, CGP 60474, CGP 74514 dihydrochloride, (R)-CR8, CVT 313, (R)-DRF053 dihydrochloride, flavopiridol, indirubin-3′-oxime, kenpaullone, LDC 000067, NSC 625987, NSC 663284, NSC 693868, NU 2058, NU 6140, NVP 2, olomoucine, [Ala⁹²]-p16 (84-103), palbociclib, PHA 767491 hydrochloride, purvalanol A, purvalanol B, R 547, ribociclib, Ro3306, roscovitine, ryuvidine, senexin A, SNS 032, SU 9516, letrozole, fulvestrant, dinaciclib, and AT7519. In certain embodiments, an agent is a CDK inhibitor selected from the group consisting of palbociclib, ribociclib, letrozole, fulvestrant, AT7519, and flavopiridol. In some embodiments, a BRAF inhibitor is vemurafenib or dabrafenib.

In some embodiments, an agent may inhibit the activation of the sympathetic nervous system. Inhibiting the activation of the sympathetic nervous system may result in decreased secretion of norepinephrine. In some aspects, agents inhibit hyper or burst activation of sympathetic nerves. In some aspects, agents inhibit secretion of norepinephrine from sympathetic nerves. In some aspects, an agent ablates sympathetic nerves. In certain embodiments, the agent that ablates sympathetic nerves is a selective neurotoxin for sympathetic nerves. In some embodiments, the selective neurotoxin is 6-hydroxy dopamine (6-OHDA) or botulinum toxin (botox). In some embodiments, an agent inhibits or blocks the release or secretion of norepinephrine, e.g., from sympathetic nerve terminals. In certain embodiments, the agent that blocks the release of norepinephrine is selected from the group consisting of guanethidine, xylocholine, bretylium, debrisoquin, and botulinum toxin.

In some embodiments, agents inhibit or block adrenergic receptor (e.g., β2 adrenergic receptors). Blocking or inhibiting adrenergic receptors, e.g., on MeSCs, prevents norepinephrine signaling. In some aspects, blocking adrenergic receptors inhibits MeSC proliferation (e.g., hyper or aberrant proliferation). In some embodiments, an agent is an adrenergic receptor blocker (e.g., a beta blocker). In some aspects, the beta blocker exhibits or has an activity that blocks an adrenergic receptor (e.g., a β2 adrenergic receptor). In some aspects, the beta blocker is non-selective or selective for an adrenergic receptor. In certain embodiments, the agent that is a adrenergic receptor blocker (e.g., a beta blocker) is selected from the group consisting of propranolol, atenolol, metoprolol, acebutolol, nadolol, sotalol, bisoprolol, penbutolol, timolol, betaxolol, labetalol, pindolol, careolol, and exmolol.

In some aspects, agents that inhibit and/or treat hair greying are inhibitors of norepinephrine secretion, inhibitors of adrenergic receptors (e.g., β2 adrenergic receptors), and/or inhibitors of MeSC hyper proliferation. In some embodiments, agents that inhibit and/or treat hair greying are CDK inhibitors and/or adrenergic receptor (e.g., β2 adrenergic receptor) inhibitors (e.g., beta blockers).

The disclosure also contemplates agents that increase and/or cause hair greying. In some embodiments, the agents increase and/or activate MeSC proliferation. In some aspects, the agent increases and/or activates sympathetic nerves. In certain aspects, the agent is norepinephrine, an Akt activator (e.g., FGF2 or SC-79), is an adrenergic beta agonist. In some aspects, the agent is delivered via Clozapine N-Oxide (CNO).

The disclosure further contemplates pharmaceutical or cosmetic compositions comprising one or more agents that inhibit and/or treat hair greying (e.g., stress-induced hair greying) in a subject. In some aspects, the pharmaceutical or cosmetic composition comprises an effective amount of one or more agents described herein for inhibiting and/or treating hair greying. In some embodiments, a pharmaceutical or cosmetic composition comprises one or more agents that inhibit MeSC proliferation (e.g., hyper or aberrant proliferation). In some embodiments, a pharmaceutical or cosmetic composition comprises one or more agents that inhibit adrenergic receptors. In some embodiments, a pharmaceutical or cosmetic composition comprises one or more agents that inhibit activation of sympathetic nerves. In some embodiments, a pharmaceutical or cosmetic composition comprises one or more agents that inhibit secretion of norepinephrine.

In some embodiments, a pharmaceutical or cosmetic composition comprises an effective amount of one or more agents for inhibiting MeSC proliferation, one or more agents for inhibiting norepinephrine secretion, and/or one or more agents for inhibiting β2 adrenergic receptors. In certain embodiments, a pharmaceutical or cosmetic composition comprises one or more CDK inhibitors. In certain embodiments, a pharmaceutical or cosmetic composition comprises one or more beta blockers. In certain embodiments, a pharmaceutical or cosmetic composition comprises one or more CDK inhibitors and one or more beta blockers.

In some embodiments, a pharmaceutical or cosmetic composition comprises an effective amount of one or more agents that inhibit MeSC proliferation, and a pharmaceutically acceptable carrier, diluent, or excipient.

The disclosure contemplates pharmaceutical or cosmetic compositions comprising one or more agents that increase and/or cause hair greying in a subject. In some aspects, the pharmaceutical or cosmetic composition comprises an effective amount of one or more agents described herein for causing and/or increasing hair greying. In some embodiments, a pharmaceutical or cosmetic composition comprises one or more agents that increase MeSC proliferation (e.g., cause hyper or aberrant proliferation). In some aspects, a pharmaceutical or cosmetic composition comprises one or more agents that activate the sympathetic nervous system. In some embodiments, a pharmaceutical or cosmetic composition comprises one or more agents that increase norepinephrine secretion (e.g., from sympathetic nerves). In some embodiments, a pharmaceutical or cosmetic composition comprises an effective amount of norepinephrine.

In some embodiments, a pharmaceutical or cosmetic composition comprises an effective amount of one or more agents that activate MeSC proliferation, and a pharmaceutically acceptable carrier, diluent, or excipient.

Pharmaceutical or cosmetic compositions described herein are for administration to a subject. These pharmaceutically acceptable compositions comprise a therapeutically-effective amount of one or more of the agents, formulated together with one or more pharmaceutically acceptable carriers (additives) and/or diluents. The pharmaceutical compositions of the present invention can be specially formulated for administration in solid or liquid form, including those adapted for the following: (1) oral administration, for example, drenches (aqueous or non-aqueous solutions or suspensions), gavages, lozenges, dragees, capsules, pills, tablets (e.g., those targeted for buccal, sublingual, and systemic absorption), boluses, powders, granules, pastes for application to the tongue; (2) parenteral administration, for example, by subcutaneous, intramuscular, intrathecal, intercranially, intravenous or epidural injection as, for example, a sterile solution or suspension, or sustained-release formulation; (3) topical application, for example, as a cream, ointment, or a controlled-release patch or spray applied to the skin; (4) transdermally; and (5) nasally. Additionally, agents can be implanted into a patient or injected using a drug delivery system. (See, for example, Urquhart, et al., Ann. Rev. Pharmacol. Toxicol. 24: 199-236 (1984); Lewis, ed. “Controlled Release of Pesticides and Pharmaceuticals” (Plenum Press, New York, 1981); U.S. Pat. Nos. 3,773,919; and 35 3,270,960, content of all of which is herein incorporated by reference.)

As used herein, the term “pharmaceutically acceptable” refers to those agents, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

As used herein, the term “pharmaceutically-acceptable carrier” means a pharmaceutically-acceptable material, composition or vehicle, such as a liquid or solid filler, diluent, excipient, manufacturing aid (e.g., lubricant, talc magnesium, calcium or zinc stearate, or steric acid), or solvent encapsulating material, involved in carrying or transporting the subject agent from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, methylcellulose, ethyl cellulose, microcrystalline cellulose and cellulose acetate; (4) powdered tragacanth; (5) malt; (6) gelatin; (7) lubricating agents, such as magnesium stearate, sodium lauryl sulfate and talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol (PEG); (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) pH buffered solutions; (21) polyesters, polycarbonates and/or polyanhydrides; (22) bulking agents, such as polypeptides and amino acids (23) serum component, such as serum albumin, HDL and LDL; (22) C₂-C₁₂ alcohols, such as ethanol; and (23) other non-toxic compatible substances employed in pharmaceutical or cosmetic formulations. Wetting agents, coloring agents, release agents, coating agents, sweetening agents, flavoring agents, perfuming agents, preservative and antioxidants can also be present in the formulation. The terms such as “excipient”, “carrier”, “pharmaceutically acceptable carrier” or the like are used interchangeably herein.

The phrase “therapeutically-effective amount” as used herein means that amount of an agent, material, or composition comprising an agent described herein which is effective for producing some desired therapeutic effect in at least a sub-population of cells in an animal at a reasonable benefit/risk ratio applicable to any medical or cosmetic treatment. For example, an amount of an agent administered to a subject that is sufficient to produce a statistically significant, measurable decrease or increase in MeSC proliferation.

The determination of a therapeutically or cosmetically effective amount of the agents and compositions disclosed herein is well within the capability of those skilled in the art. Generally, a therapeutically effective amount can vary with the subject's history, age, condition, sex, and the administration of other pharmaceutically active agents.

In general, a therapeutically or cosmetically effective amount can be administered in one or more administrations, applications, or dosages. The therapeutically or cosmetically effective amount of a formulation depends on the specific anti-greying formulation selected. For example, the compositions or agents may be administered from one or more times per day to one or more times per week; including one to five times per day, e.g., once, twice, or three times every day, or one to five times every other day, e.g., once, twice, or three times every other day. One of skill in the art will appreciate that certain factors may influence the dosage and timing required to effectively treat a subject, including, but not limited to, the severity of the greying hair, any previous treatments, the general health and/or age of the subject, and any diseases present. Treatment may comprise a single treatment or a series of treatments.

Dosage, toxicity, and cosmetic or therapeutic efficacy of the formulations can be determined by standard procedures in cell cultures or experimental animals, e.g., for determining the ED50 (the cosmetically effective dose in 50% of the population). The data obtained from cell culture assays and animal studies can be used in formulating a range of dosages for use in humans. The dosage may vary within this range depending upon the dosage form employed and the route of administration utilized. For any formulation or composition used in the methods of reducing hair greying described herein, the cosmetically or prophylactically effective dose can be estimated initially from cell culture assays.

Hair care compositions or agents can be applied, e.g., twice daily, daily, every other day, twice weekly, biweekly, or monthly. In some embodiments, after a period of daily (or more frequent) application, the compositions or agents can be applied less frequently, as needed to maintain efficacy.

As used herein, the term “administer” refers to the placement of an agent or composition into or on a subject (e.g., a subject in need) by a method or route which results in at least partial localization of the agent or composition at a desired site such that desired effect is produced. Routes of administration suitable for the methods of the invention include both local and systemic routes of administration. Generally, local administration results in more of the administered agents being delivered to a specific location as compared to the entire body of the subject, whereas, systemic administration results in delivery of the agents to essentially the entire body of the subject.

The compositions and agents disclosed herein can be administered by any appropriate route known in the art including, but not limited to, oral or parenteral routes, including intravenous, intramuscular, subcutaneous, transdermal, airway (aerosol), pulmonary, nasal, rectal, and topical (including buccal and sublingual) administration. Exemplary modes of administration include, but are not limited to, injection, infusion, instillation, inhalation, or ingestion. “Injection” includes, without limitation, intravenous, intramuscular, intraarterial, intrathecal, intraventricular, intracranial, intracapsular, intraorbital, intracardiac, intradermal, intraperitoneal, transtracheal, subcutaneous, subcuticular, intraarticular, sub capsular, subarachnoid, intraspinal, intracerebro spinal, and intrastemal injection and infusion. In preferred embodiments of the aspects described herein, the compositions are administered by topical application.

For topical application, the compositions and agents are formulated into solutions, suspensions, lotions, sprays, shampoos, hair conditions, serums, patches, wipes, gels, hydrogels, powders, patches, impregnated pads, emulsions, vesicular dispersions, sprays, aerosols, foams, ointments, tinctures, salves, gels, cleansing soaps, cleansing cakes, or creams as generally known in the art. The formulation can be, e.g., in a multi-use or single-use applicator. Topical administration can include the application of the pharmaceutical or cosmetic compositions to the scalp and/or hair.

As used herein, a “subject” means a human or animal (e.g., a mammal). Usually the animal is a vertebrate such as a primate, rodent, domestic animal or game animal. Primates include chimpanzees, cynomologous monkeys, spider monkeys, and macaques, e.g., Rhesus. Rodents include mice, rats, woodchucks, ferrets, rabbits and hamsters. Domestic and game animals include cows, horses, pigs, deer, bison, buffalo, feline species, e.g., domestic cat, canine species, e.g., dog, fox, wolf, avian species, e.g., chicken, emu, ostrich, and fish, e.g., trout, catfish and salmon. Patient or subject includes any subset of the foregoing, e.g., all of the above, but excluding one or more groups or species such as humans, primates or rodents. In certain embodiments of the aspects described herein, the subject is a mammal, e g , a primate, e.g., a human. The terms, “patient” and “subject” are used interchangeably herein. A subject can be male or female. In some embodiments the subject suffers from hair greying, e.g., stress-induced hair greying.

Biomarkers and Screening Methods

In some aspects the disclosure contemplates the use of c-FOS as a biomarker for activation (e.g., burst activation) of sympathetic neurons. For example, increased levels of c-FOS may be used as a biomarker for activation of sympathetic neurons. In some aspects, increased levels of c-FOS are used as a biomarker for increased MeSC proliferation and/or for hair greying (e.g., stress-induced hair greying).

In some aspects, the disclosure also contemplates the use of phospho-histone H3 (i.e., an M phase marker) as a biomarker for MeSC proliferation. For example, an M phase marker, e.g., phospho-histone H3, may be used as a biomarker for increased MeSC proliferation. In some aspects, an upregulated M phase marker, e.g., phospho-histone H3, is used as a biomarker for hair greying (e.g., stress-induced hair greying).

In some aspects, the disclosure also contemplates the use of a cell cycle regulator, e.g., cyclin-dependent kinase 2 (Cdk2), as a biomarker for MeSC proliferation. For example, an upregulated cell cycle regulator, e.g., Cdk2, may be used as a biomarker for increased MeSC proliferation. In some aspects, an upregulated cell cycle regulator, e.g., Cdk2, is used as a biomarker for hair greying (e.g., stress-induced hair greying).

In some aspects, the disclosure also contemplates the use of receptors for ligands that promote MeSC proliferation, differentiation, and migration (e.g., c-Kit and Mc1r) as biomarkers for MeSC proliferation. For example, an upregulated receptor, e.g., c-Kit and/or Mc1r, may be used as a biomarker for increased MeSC proliferation. In some aspects, an upregulated receptor, e.g., c-Kit and/or Mc1r, is used as a biomarker for hair greying (e.g., stress-induced hair greying).

In some aspects, the disclosure also contemplates the use of genes involved in melanogenesis (e.g., Mitf, Tyrp1, Tyr, Oca2, and/or Pmel) as biomarkers for MeSC proliferation. For example, an upregulated melanogenesis gene, e.g., Mitf, Tyrp1, Tyr, Oca2, and/or Pme1, may be used as a biomarker for increased MeSC proliferation. In some aspects, an upregulated melanogenesis gene, e.g., Mitf, Tyrp1, Tyr, Oca2, and/or Pme1, is used as a biomarker for hair greying (e.g., stress-induced hair greying).

The disclosure also contemplates assays for detecting hair greying (e.g., stress-induced hair greying) in a subject. In some embodiments, an assay includes obtaining a sample (e.g., a hair follicle) from a subject, and determining if the sample includes increased levels of one or more of c-FOS, phospho-histone H3, Cdk2, c-Kit, Mc1r, Mitf, Tyrp1, Tyr, Oca2, and Pme1, as compared to a reference sample. Increased levels of one or more of c-FOS, phospho-histone H3, Cdk2, c-Kit, Mc1r, Mitf, Tyrp1, Tyr, Oca2, and Pme1, may be an indicator of increased MeSC proliferation, and thus an indicator of hair greying.

The disclosure also contemplates an assay for screening potential active agents that reduce hair greying (e.g., stress-induced hair greying). In some embodiments, the assay includes inducing oxidative stress, e.g., by the application of an H₂O ₂ solution or ionizing radiation, and topically administering the test agents to human primary hair follicles and detecting one or more of a reduction in oxidative damage, a reduction in differentiation of the MeSC differentiation into pigmented cells, and a reduction in the greying of the hair. In general, the primary hair follicles are grown and maintained in culture and the oxidative stress can be determined before, during, and after treatments with the test agent. A negative and a positive control reference may be untreated hair follicles and hair follicles treated with a standard antioxidant, respectively.

In some embodiments, the assay methods of screening for agents that reduce or inhibit hair greying include isolating follicles from a human specimen, embedding them in a semisolid media, and topically administering the oxidative stress-inducing agent and the test active agent, in any order or simultaneously, or at different time intervals between administrations, e.g., once daily, twice daily, or 5, 10, 15, 20 or more minutes apart, or 1, 2, 3, 4, 5 or more hours apart, or 1, 2, 3, 4 or more days apart. The assay methods described herein can further comprise, for example, detecting or imaging pigmented cells in the specimen or measuring the level of oxidative activity in comparison to a reference specimen. Methods for detecting or imaging cells, and measuring levels of oxidative activity are known in the art.

In some embodiments, a test compound is applied to a test sample comprising living follicles, e.g., human follicles, and one or more effects of the test compound is evaluated using the assay described herein. In some embodiments, the ability of the test compound to promote cell viability, reduce apoptosis, or reduce the generation or levels of reactive oxygen species is assayed.

Methods for evaluating each of these effects are known in the art. For example, the ability to promote cell viability and/or reduce apoptosis can be evaluated, e.g., by detecting cell numbers or proliferation using manual or automated cell counting, by detecting ratios of live/dead cells, or by detecting apoptotic processes (e.g., breakdown of the nucleus, increase in cell membrane permeability, chromatin condensation, protease activity (e.g., caspase activity), disruption of active mitochondria, or increases in autophagy). A number of commercially available assays (e.g., from life technologies) can be used in these methods. See, e.g., The Molecular Probes® Handbook, Assays for Cell Viability, Proliferation and Function—Chapter 15 (2010).

The ability of an agent to modulate ROS generation can be evaluated, e.g., using fluorescent and chemiluminescent assays with reagents such as 2′,7′-dichlorodihydrofluorescein diacetate (H2DCFDA); 5-(and 6-)carboxy-2′,7′-difluorodihydrofluorescein diacetate (carboxy-H2DFFDA) and CM-H2DCFDA, derivatives of H2DCFDA; 3′-(p-aminophenyl) fluorescein (APF) and 3′-(p-hydroxyphenyl) fluorescein (HPF); dihydrocalcein AM; OxyBURST® Green assay reagent; Dihydrorhodamine 123; or CellROX® reagents for oxidative stress detection, all of which are commercially available. See, e.g., The Molecular Probes® Handbook, Probes for Reactive Oxygen Species, Including Nitric Oxide—Chapter 18; Generating and Detecting Reactive Oxygen Species—Section 18.2 (2010).

The ability to modulate expression of a protein can be evaluated at the gene or protein level, e.g., using quantitative PCR or immunoassay methods. In some embodiments, high throughput methods, e.g., protein or gene chips are known in the art (see, e.g., Ch. 12, Genomics, in Griffiths et al., Eds. Modern Genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect an effect on the expression of a protein at the gene or protein level.

As used herein, “small molecules” refers to small organic or inorganic molecules of molecular weight below about 3,000 Daltons. In general, small molecules useful for the invention have a molecular weight of less than 3,000 Daltons (Da). The small molecules can be, e.g., from at least about 100 Da to about 3,000 Da (e.g., between about 100 to about 3,000 Da, about 100 to about 2500 Da, about 100 to about 2,000 Da, about 100 to about 1,750 Da, about 100 to about 1,500 Da, about 100 to about 1,250 Da, about 100 to about 1,000 Da, about 100 to about 750 Da, about 100 to about 500 Da, about 200 to about 1500 Da, about 500 to about 1000 Da, about 300 to about 1000 Da, or about 100 to about 250 Da).

The test compounds can be, e.g., natural products or members of a combinatorial chemistry library. A set of diverse molecules should be used to cover a variety of functions such as charge, aromaticity, hydrogen bonding, flexibility, size, length of side chain, hydrophobicity, and rigidity. Combinatorial techniques suitable for synthesizing small molecules are known in the art, e.g., as exemplified by Obrecht and Villalgordo, Solid-Supported Combinatorial and Parallel Synthesis of Small-Molecular-Weight Compound Libraries, Pergamon-Elsevier Science Limited (1998), and include those such as the “split and pool” or “parallel” synthesis techniques, solid-phase and solution-phase techniques, and encoding techniques (see, for example, Czarnik, Curr. Opin. Chem. Bio. 1:60-6 (1997)). In addition, a number of small molecule libraries are commercially available. A number of suitable small molecule test compounds are listed in U.S. Pat. No. 6,503,713, incorporated herein by reference in its entirety.

Libraries screened using the methods of the present invention can comprise a variety of types of test compounds. A given library can comprise a set of structurally related or unrelated test compounds. In some embodiments, the test compounds are peptide or peptidomimetic molecules. In some embodiments, the test compounds are nucleic acids.

In some embodiments, the test compounds and libraries thereof can be obtained by systematically altering the structure of a first test compound, e.g., a first test compound that is structurally similar to a known natural binding partner of the target polypeptide, or a first small molecule identified as capable of binding the target polypeptide, e.g., using methods known in the art or the methods described herein, and correlating that structure to a resulting biological activity, e.g., a structure-activity relationship study. As one of skill in the art will appreciate, there are a variety of standard methods for creating such a structure-activity relationship. Thus, in some instances, the work may be largely empirical, and in others, the three-dimensional structure of an endogenous polypeptide or portion thereof can be used as a starting point for the rational design of a small molecule compound or compounds. For example, in one embodiment, a general library of small molecules is screened, e.g., using the methods described herein.

In some embodiments, the test sample is, or is derived from (e.g., a sample taken from), a human having grey hair (e.g., hair that has greyed due to normal aging, early onset greying, or due to exposure to conditions that promote hair greying); normally aging hair that is not yet grey, or a known condition that affects hair greying (e.g., strictly segmental vitiligo (SSV) and non-segmental vitiligo (NSV)); or an in vivo model of a condition that affects hair greying. For example, an animal model of hair greying, e.g., a rodent such as a mouse (e.g., a Bcl-2 knockout mouse as described in Veis et al., Cell 75, 229 (1993), or Mitf^{vit/vit} mice as described in Lerner et al., J. Invest. Dermatol. 87, 299 (1986)), can be used.

A test compound that has been screened by a method described herein and determined to promote cell viability, reduce apoptosis, or reduce the generation or levels of reactive oxygen species in an assay described herein (e.g., an ex vivo follicular assay), can be considered a candidate anti-greying compound. In some embodiments a candidate anti-greying compound can be screened, e.g., in an in vivo model of a disorder, e.g., on a model of mammalian hair greying, or in a human, and determined to have a desirable effect on aging hair or greying hair, e.g., on delaying, inhibiting, or reducing hair greying.

In some embodiments, methods for screening agents (test molecules), e.g., plant extracts, polypeptides, polynucleotides, or inorganic or organic large or small molecule compounds, comprises identifying agents useful in reducing hair greying.

Selected compounds can be optionally improved and/or derivatized, and formulated with dermatologically acceptable carriers to form hair care formulations as described herein. For example, test compounds identified as “hits” (e.g., test compounds that promote cell viability, reduce apoptosis, or reduce the generation or levels of reactive oxygen species in an assay as described herein) can be selected and systematically altered, e.g., using rational design, to optimize binding affinity, avidity, specificity, non-toxicity, tolerability, hydrophilicity, hydrophobicity, or other parameter. Such improvements can also be screened using the methods described herein. Thus, in some embodiments, the methods includes screening a first library of compounds using an assay method described herein, identifying one or more hits in that library, subjecting those hits to systematic structural alteration to create a second library of compounds structurally related to the hit, and screening the second library using the assay methods described herein or other methods known in the art.

Test compounds and mixtures identified as hits can be considered for cosmetically or pharmaceutically effective formulations, useful in reducing or inhibiting the greying of hair. A variety of techniques useful for determining the structures of “hits” can be used in the methods described herein, e.g., NMR, mass spectrometry, chromatography equipped with electron capture detectors, fluorescence and absorption spectroscopy. The invention also includes compounds identified as “hits” by the methods described herein, and methods for their administration and use in the treatment, prevention, or delay of development or progression of the greying of hair.

### EXEMPLIFICATION

### Example 1

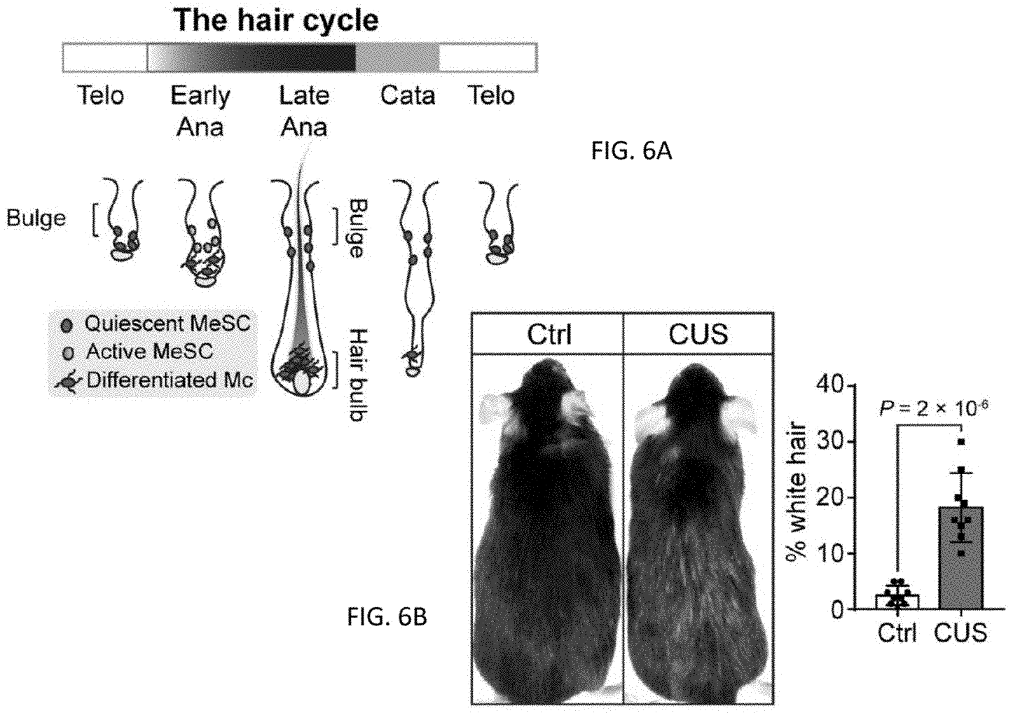

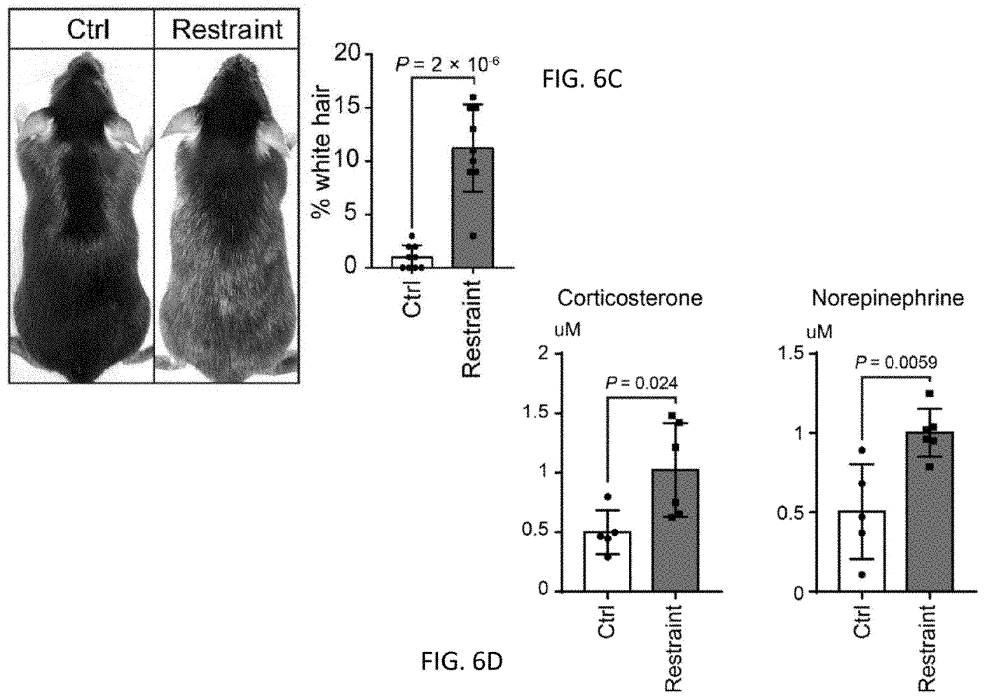

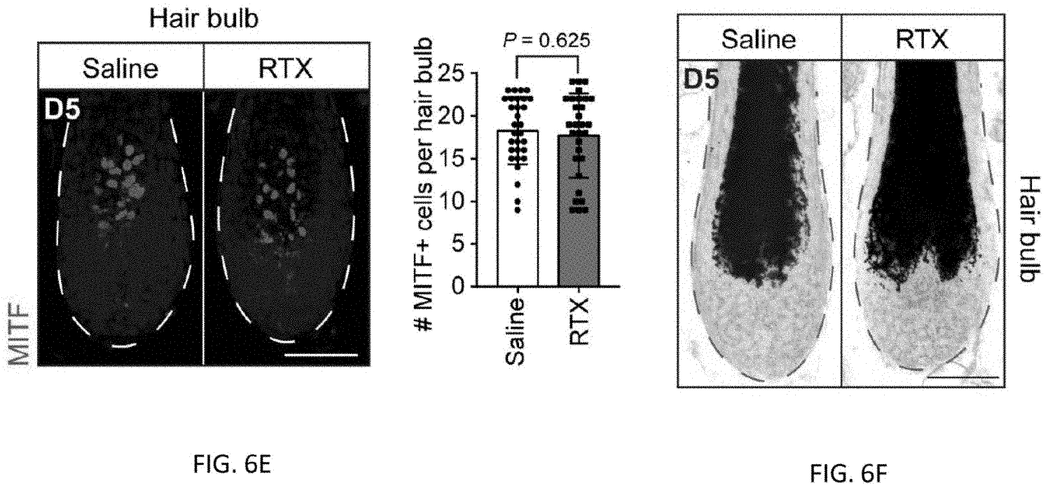

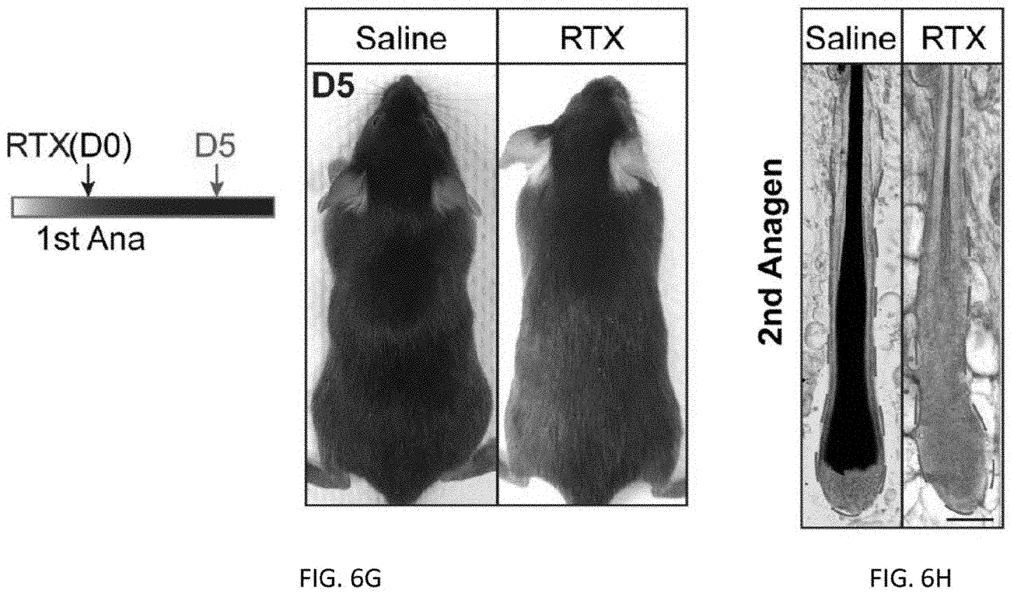

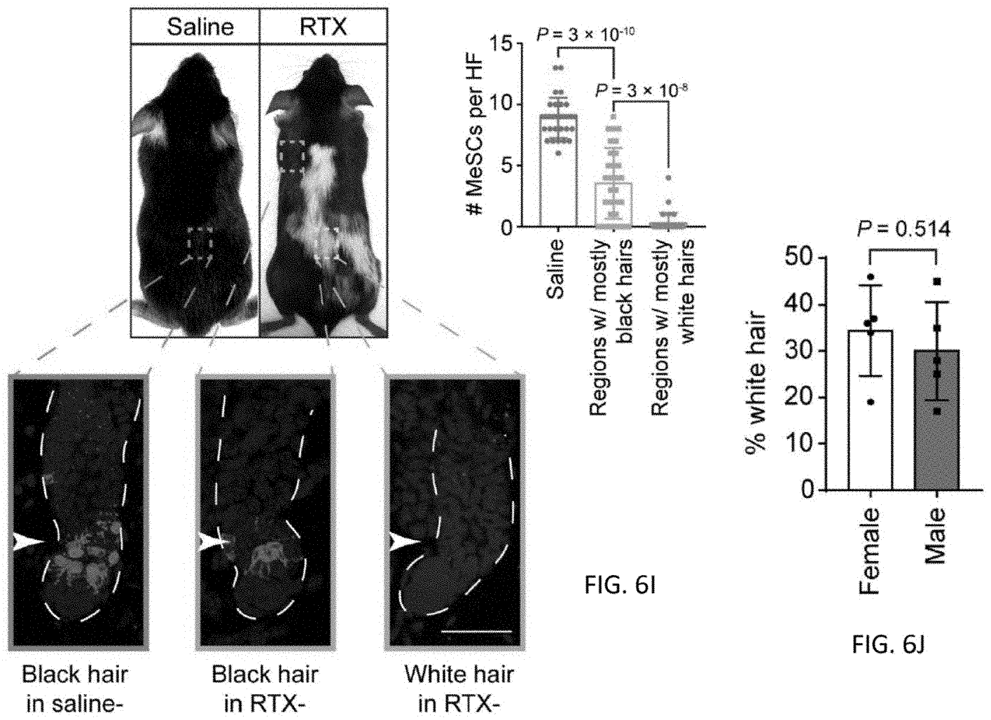

Stress has been anecdotally associated with diverse tissue changes including hair greying. However, whether external stressors indeed are the causal factors, and if stress-related changes occur at the level of somatic stem cells, remain poorly understood. The hair follicle cycles between growth (anagen), degeneration (catagen), and rest (telogen) [5]. The bulge and hair germ region harbors two stem cell populations—epithelial-derived hair follicle stem cells (HFSCs) and neural crest-derived MeSCs [6]. HFSCs and MeSCs are normally quiescent except during early anagen, when HFSCs and MeSCs are activated concurrently to regenerate a pigmented hair [7, 8]. Activation of HFSCs produces a new hair follicle. Activation of MeSCs generates differentiated melanocytes that migrate downward, while MeSCs remain close to the bulge. At the hair bulb, differentiated melanocytes synthesize melanin to color the newly regenerated hair from the root. At catagen, mature melanocytes are destroyed, leaving only the MeSCs that will initiate new rounds of melanogenesis in future cycles (FIG. 6A) [9, 10]. The stereotypic behaviour of MeSCs and melanocytes, as well as the visible nature of hair colour, makes the melanocyte lineage an accessible model to investigate how stress influences tissue regeneration.

Diverse Stressors Induce Hair Greying

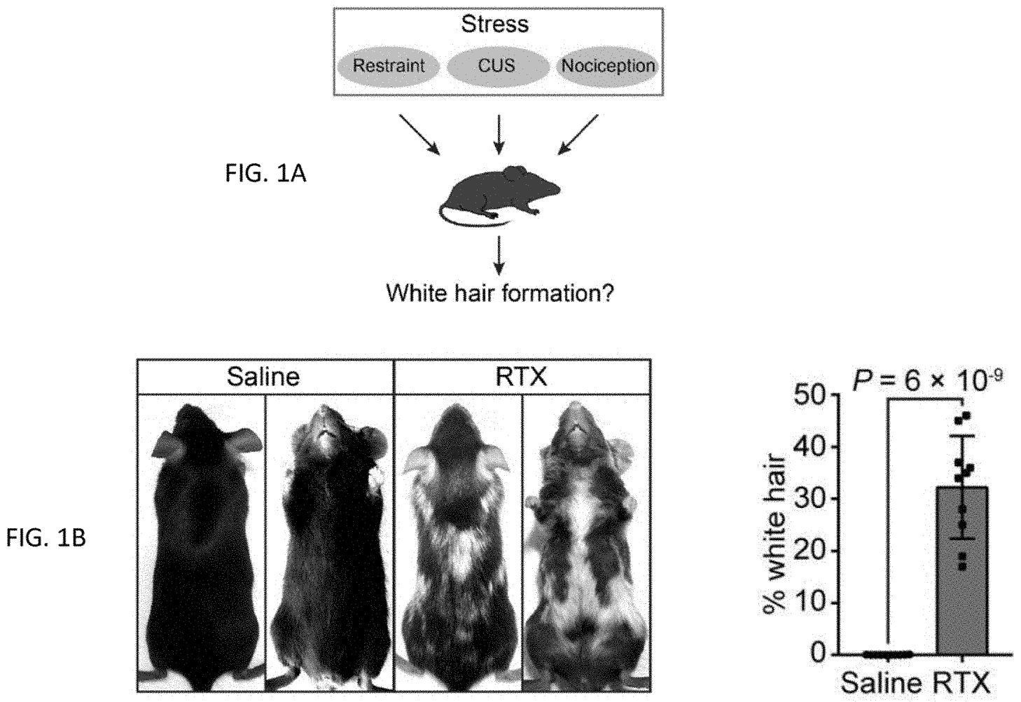

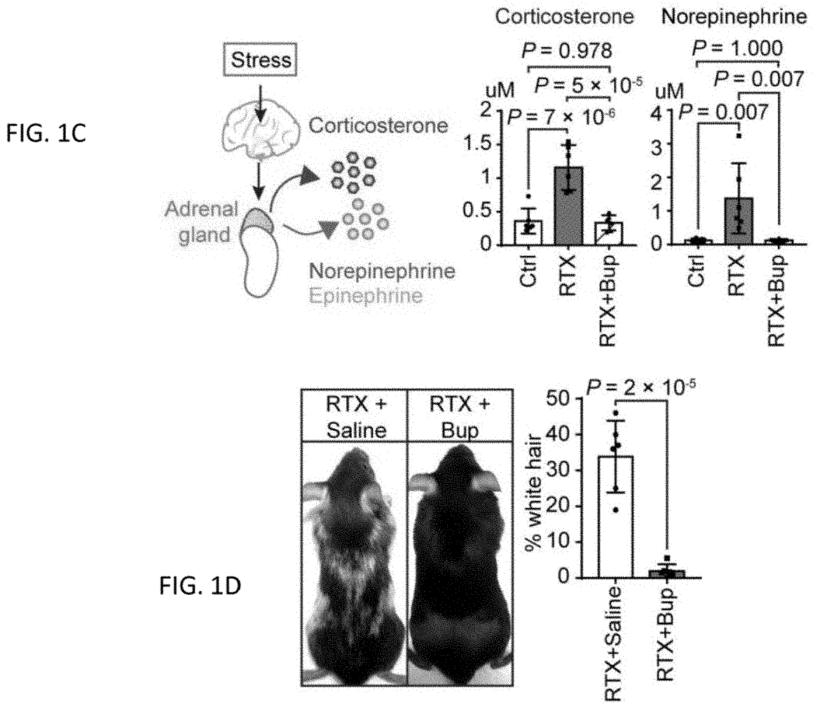

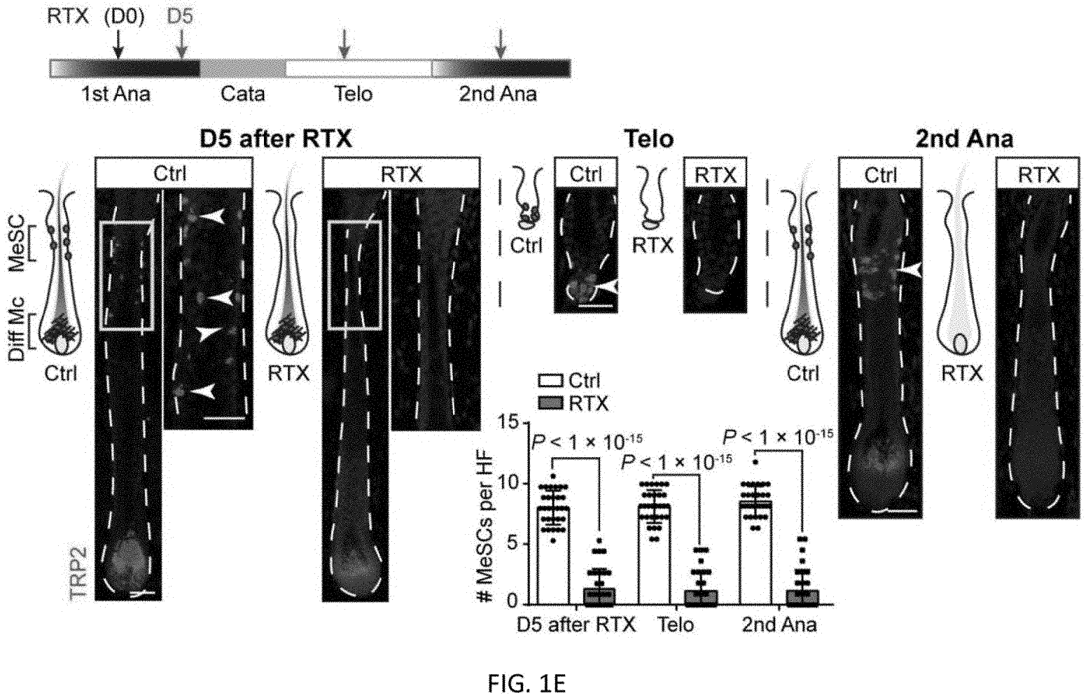

To examine whether psychological or physical stressors promote hair greying, three approaches to model stress in black coat colour C57BL/6J mice were used: restraint stress [11, 12], chronic unpredictable stress [13, 14], and nociception-induced stress via injection of resiniferatoxin (RTX, a capsaicin analogue) [15, 16]. All three procedures led to increased numbers of unpigmented white hairs over time. Restraint stress and chronic unpredictable stress led to noticeable hair greying after 3-5 rounds of hair cycles. Nociception-induced stress produced the most pronounced and rapid effect—many new hairs formed in the next hair cycle following RTX injection became unpigmented (FIGS. 1A-1B, FIGS. 6B-6C).

Psychological or physical stressors trigger the adrenal glands to release stress hormones and catecholamines into the bloodstream [17]. Indeed, an increase in both corticosterone (cortisol equivalent in rodents; a stress hormone) and norepinephrine (a catecholamine) was detected in the blood of mice subjected to different stressors (FIG. 1C, FIG. 6D), suggesting that the approaches induced classical stress responses.

RTX induces nociception by activating nociceptive sensory neurons [18]. Blocking the ability of an animal to sense pain with buprenorphine (an opioid analgesia) prevents the increase of corticosterone and norepinephrine after RTX injection, suggesting that blocking pain sensation alleviates the physiological stress responses induced by RTX (FIG. 1C). Moreover, buprenorphine also suppressed white hair formation in RTX-injected animals (FIG. 1D). These data show that regardless of stress modality, premature hair greying can occur under stress. Because the effect of nociception induction on hair greying was the strongest and most rapid of all stressors tested, a focus was placed on RTX injection as the primary stressor.

### Stress Leads to Loss of MeSCs

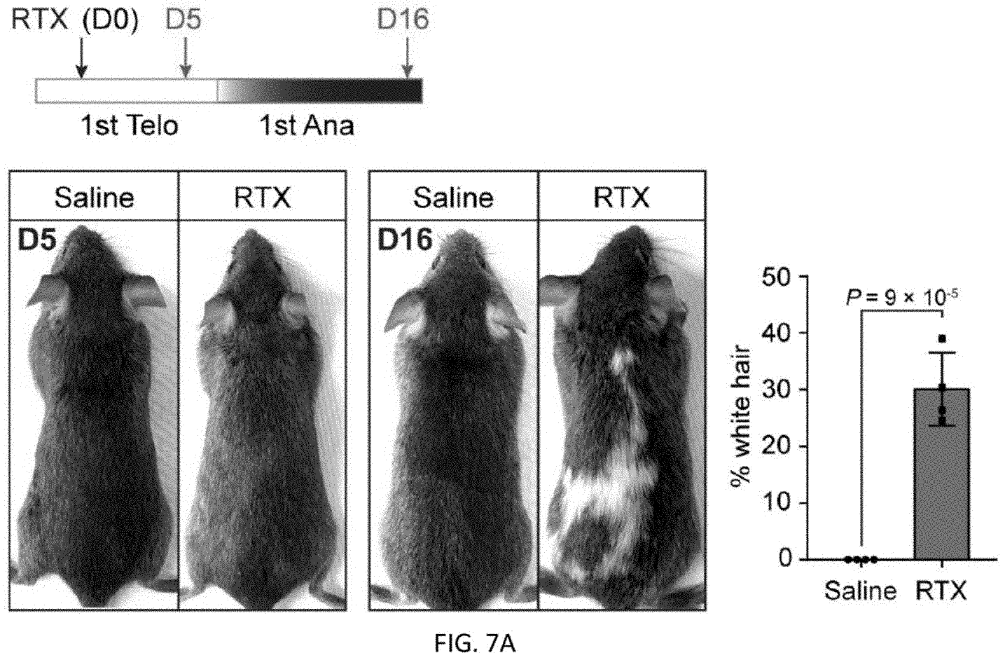

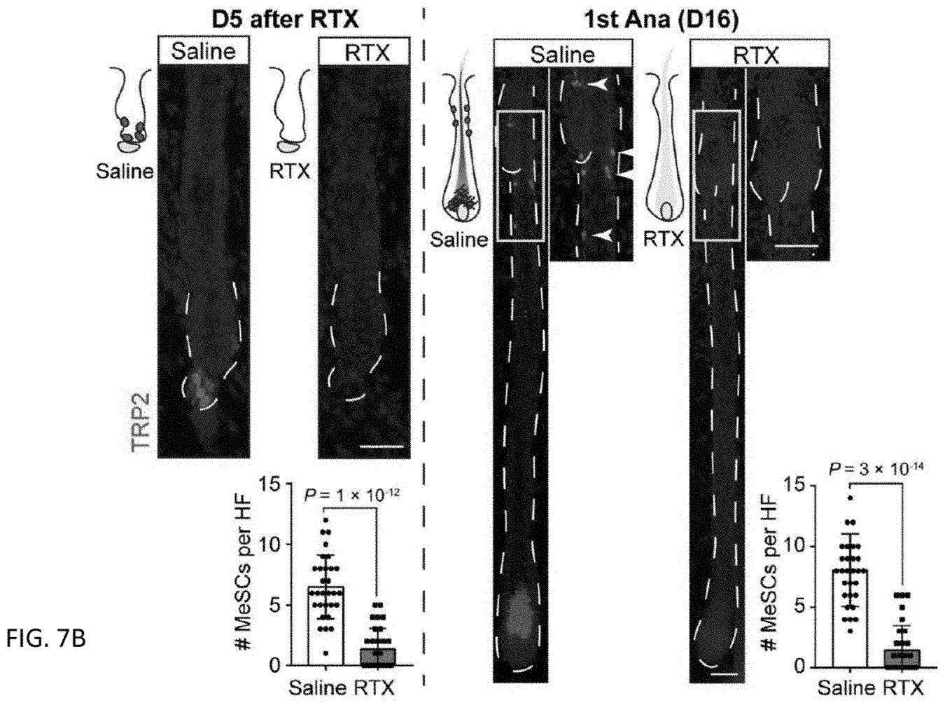

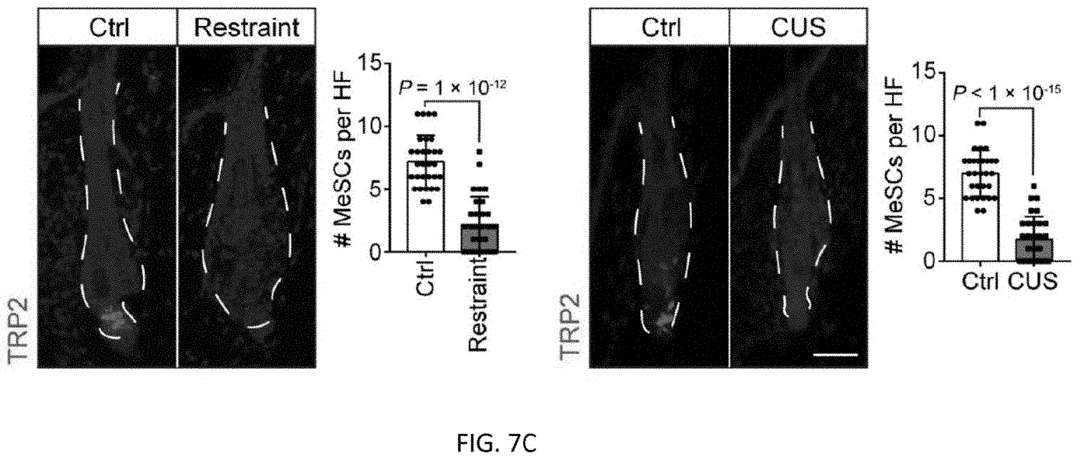

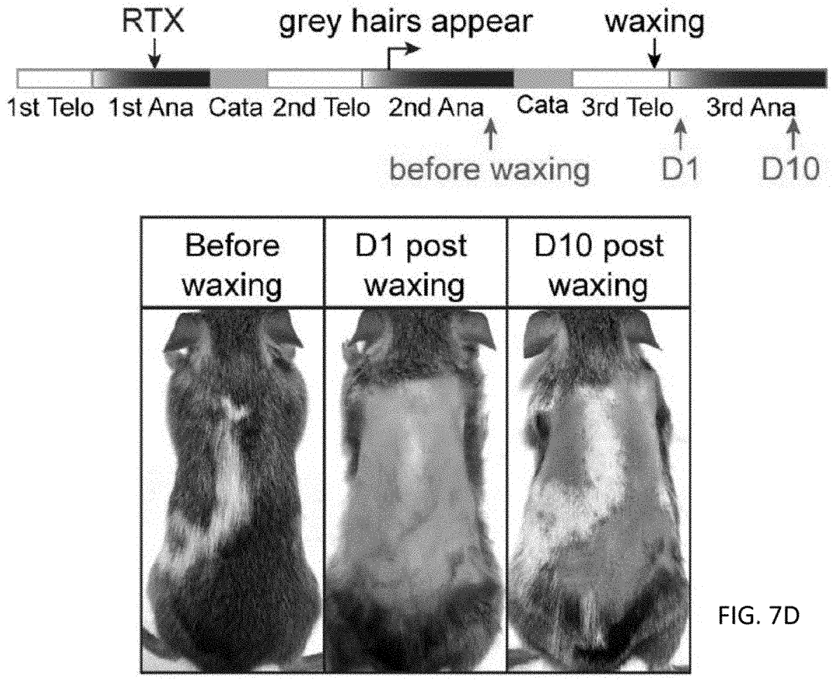

Loss of hair pigmentation can be due to defects in melanin synthesis [19, 20], loss of differentiated melanocytes [21], or problems in MeSC maintenance [22]. To understand how stress impacts the melanocyte lineage, RTX was injected into mice in anagen, when both MeSCs and differentiated melanocytes were present but located within distinct compartments—MeSCs were near to the bulge while differentiated melanocytes were at the hair bulb (FIG. 1E). Upon RTX injection, TRP2+MeSCs were significantly reduced across the entire skin (FIG. 1E, bar graph). In many hair follicles, MeSCs were lost completely from the bulge within 5 days, while differentiated melanocytes in the same hair follicle remained unperturbed (FIG. 1E, D5 after RTX, FIG. 6E). These differentiated melanocytes continued to generate pigments, and the hair coat remained black (FIGS. 6F-6G). When hair follicles in the RTX-injected animals entered catagen and telogen, many have lost all MeSCs (FIG. 1E, Telo). Subsequently, when the next round of anagen initiated, differentiated melanocytes were not produced to color new hair shafts, and unpigmented hairs emerged (FIG. 1E, 2nd Ana, FIG. 6H). Although some regenerated hairs remained pigmented, the MeSC numbers in these pigmented hairs were also reduced (FIG. 6I). RTX injection led to the same extent of hair greying in both male and female mice (FIG. 6J). Moreover, RTX also caused MeSC loss when injected during telogen. In this case, unpigmented hairs appeared as soon as new hairs emerged in the following anagen (FIGS. 7A-7B). These results suggest that MeSCs are exquisitely sensitive to RTX-induced stress, while differentiated melanocytes or melanin synthesis are not directly affected. MeSCs were also lost or reduced in mice subjected to restraint stress or chronic unpredictable stress (FIG. 7C). Since stress depleted MeSCs, the loss of hair pigmentation in all three conditions was permanent (FIG. 7D). Collectively, these data indicate that stress leads to the loss of MeSCs.

### Norepinephrine Drives MeSC Loss

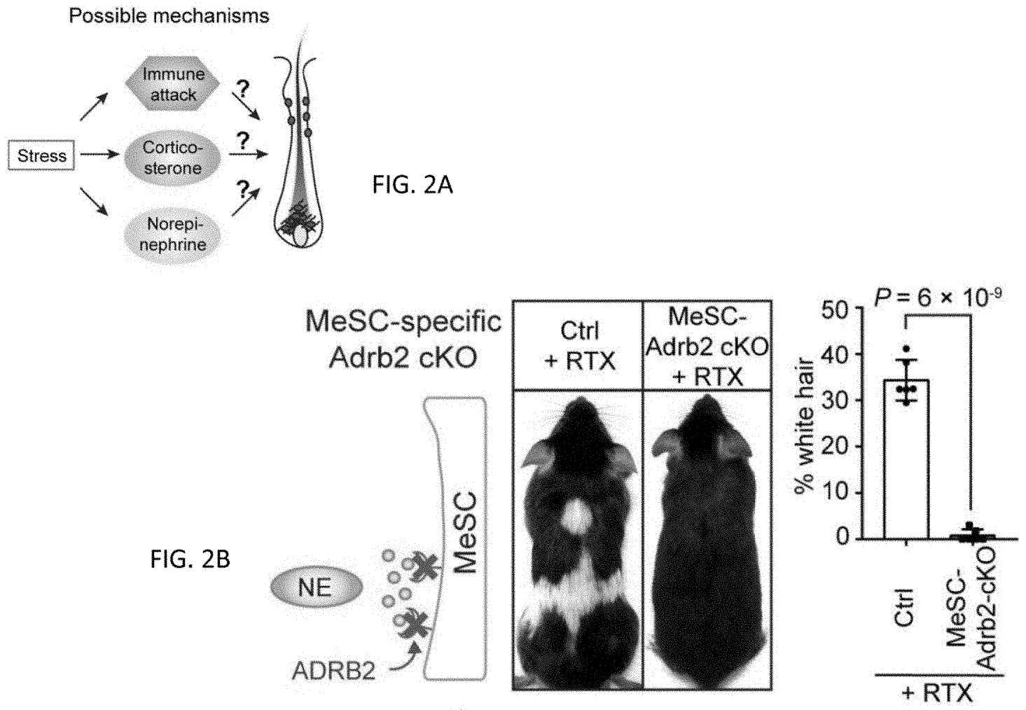

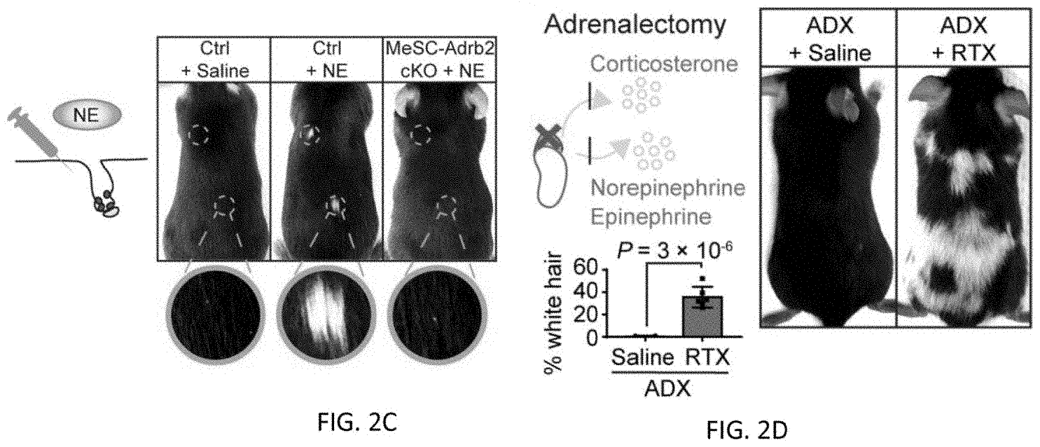

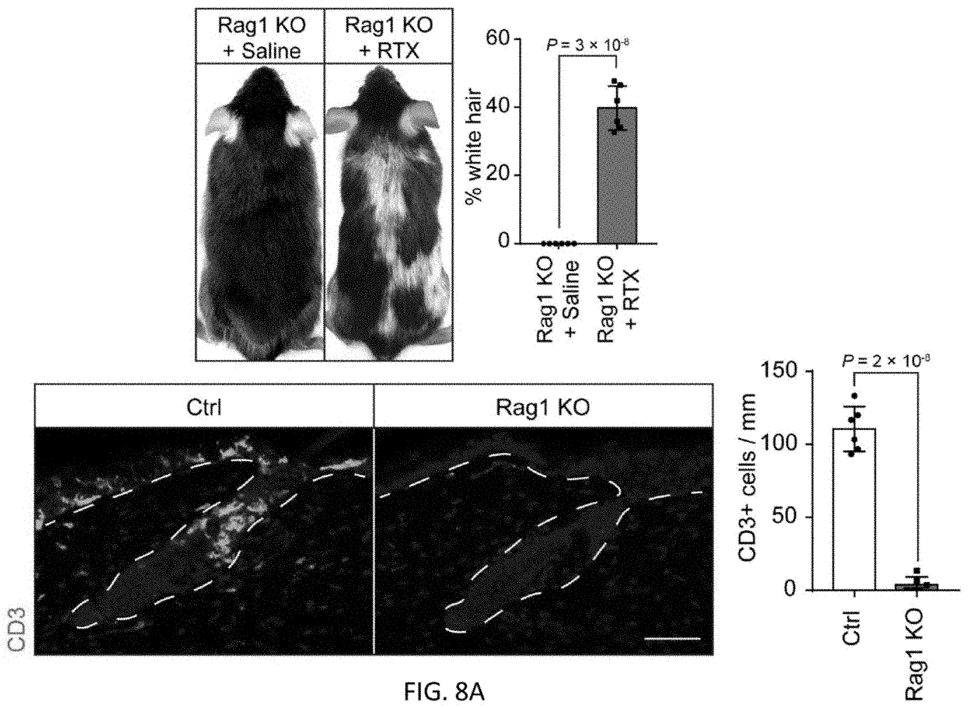

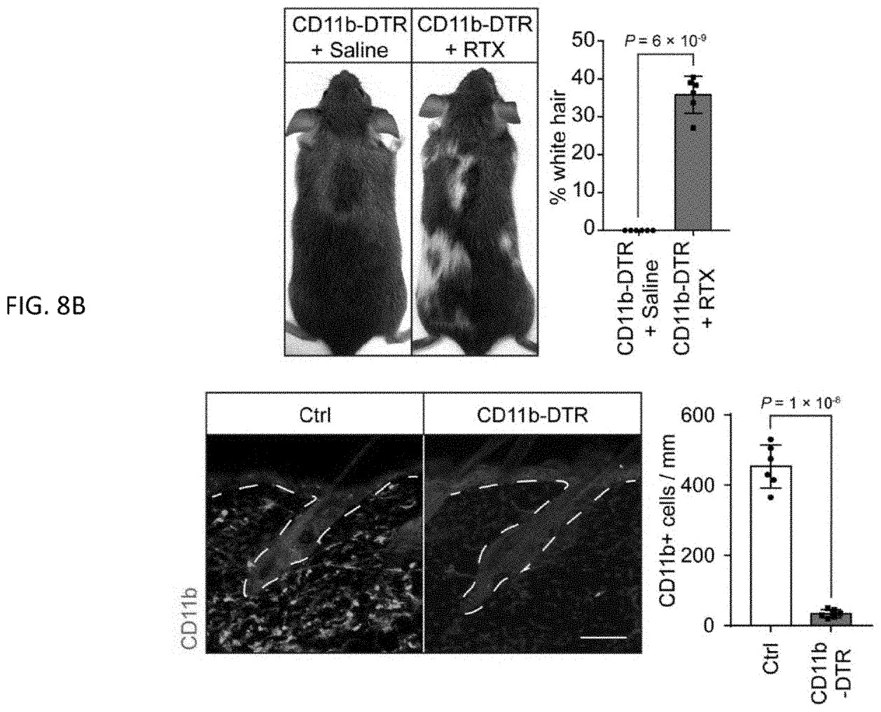

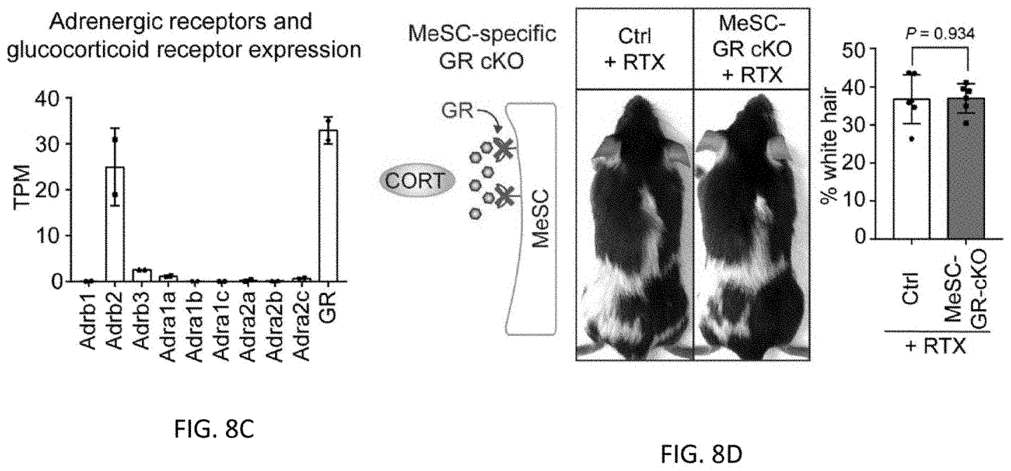

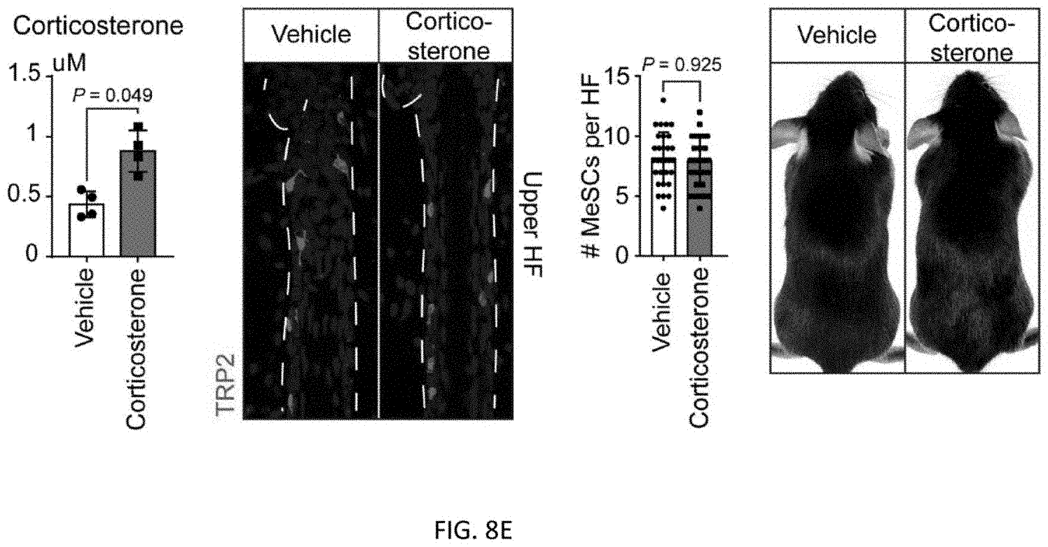

Next, how stress transmits to the periphery to alter MeSCs was examined (FIG. 2A) Immune attack has been postulated to cause stress-induced hair greying [2, 23]. To test the involvement of the immune system, RTX was injected into Ragl mutant mice, which lack both T and B cells, and into CD11b-DTR mice, in which myeloid lineages had been ablated by diphtheria toxin. Injection of RTX into these immune-deficient mice still resulted in white hair formation, suggesting that RTX-induced hair greying is independent of T cells, B cells, or myeloid cells (FIGS. 8A-8B).

Since all stressors led to elevated corticosterone and norepinephrine in the blood, it was considered if these stress-induced circulating factors played a role in stress-induced MeSC loss. The RNA sequencing (RNA-seq) data on FACS-purified MeSCs suggested that MeSCs express the glucocorticoid receptor (GR, a receptor for corticosterone) and the β2 adrenergic receptor (Adrb2, a receptor for norepinephrine) (FIG. 8C, see methods). To determine if GR mediated the effects of stress on MeSCs, GR was depleted in MeSCs using Tyr-CreER [8, 24-26]. RTX injection into Tyr-CreER; GR f1/f1 animals still resulted in hair greying (FIG. 8D). Moreover, no changes in MeSCs or hair pigmentation were observed when corticosterone was elevated via feeding (FIG. 8E). These data suggest that corticosterone is not a major driver of stress-induced MeSC loss.

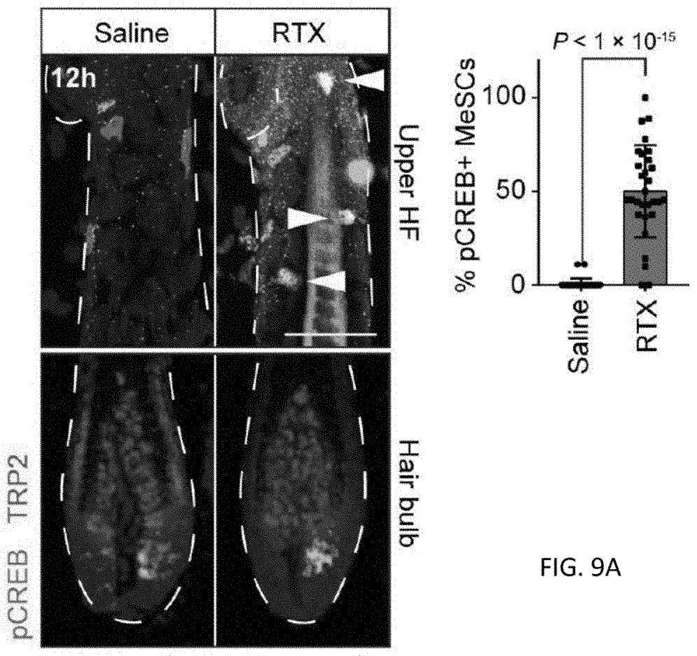

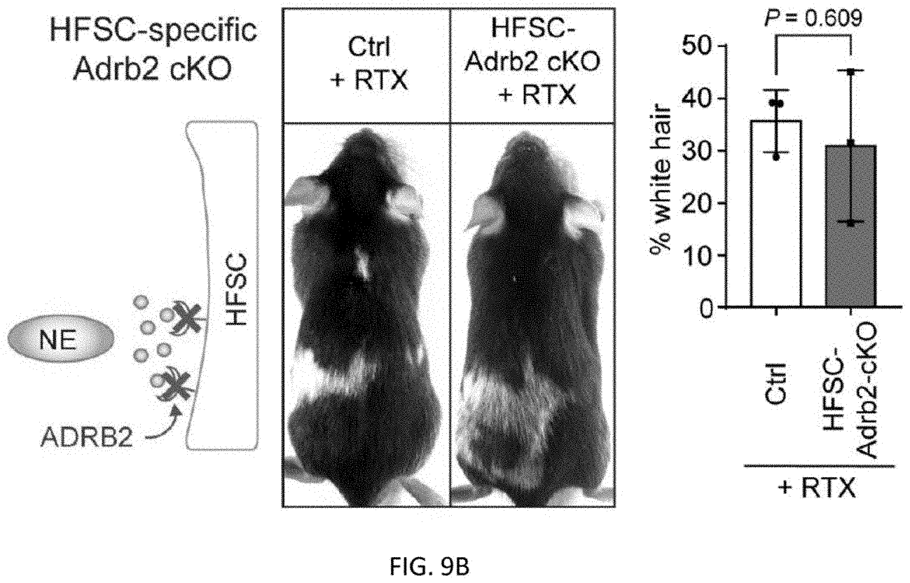

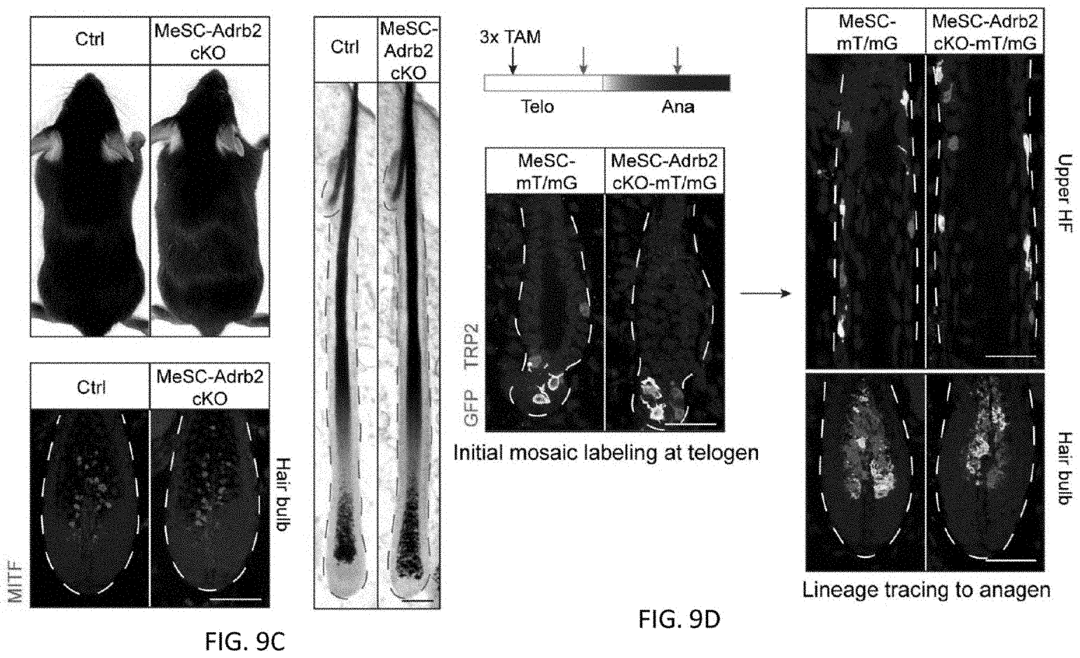

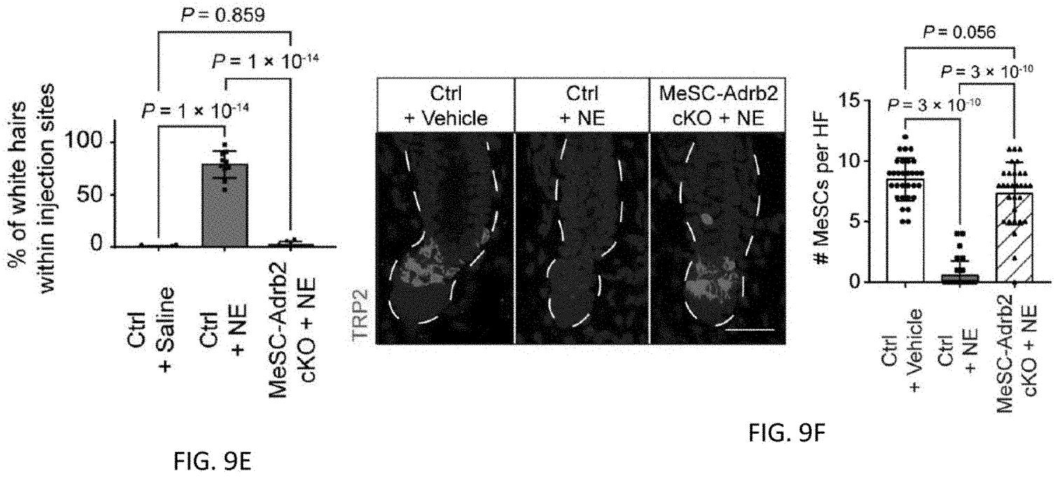

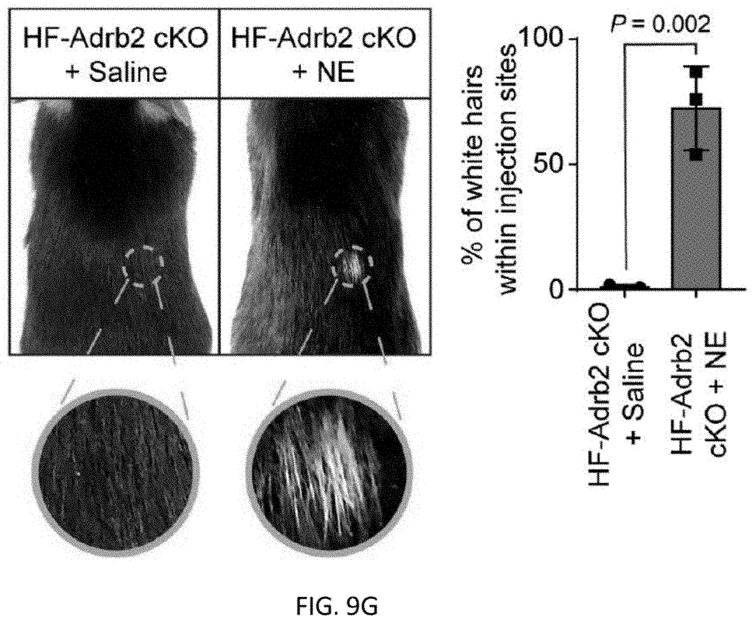

It was then considered whether ADRB2 might mediate the impact of stress on MeSCs. Upon RTX injection, a marked induction of Phospho-CREB (a downstream effector of ADRB2) was observed in MeSCs but not mature melanocytes (FIG. 9A). Moreover, when ADRB2 was depleted from MeSCs using Tyr-CreER, white hairs failed to form following RTX injection (FIG. 2B). These data suggest that ADRB2 expressed by MeSCs is essential for stress-induced hair greying. By contrast, when ADRB2 was depleted from hair follicle stem cells that share the same niche with MeSCs, RTX injection still resulted in hair greying (FIG. 9B). In the absence of stress, depletion of ADRB2 in MeSCs did not lead to changes in MeSCs, melanocytes, or pigment production, suggesting that the norepinephrine-ADRB2 pathway is dispensable for melanogenesis during the normal hair cycle (FIGS. 9C-9D). Collectively, these data suggest that norepinephrine signals through ADRB2 on MeSCs to mediate stress-induced hair greying.

To test if elevated norepinephrine was sufficient to cause hair greying in the absence of stress, norepinephrine was introduced locally to the skin via intradermal injections. Local norepinephrine injection promoted hair greying at the injection sites in wild type and in HFSC-specific adrb2 knockout mice, but failed to cause hair greying in MeSC-specific adrb2 knockout mice (FIG. 2C, FIGS. 9E-9G). Altogether, the data demonstrates that while immune cells and corticosterone are dispensable, norepinephrine signalling appears to be necessary for stress-induced hair greying and sufficient to trigger hair greying in the absence of stress.

### Finding the Source of Norepinephrine

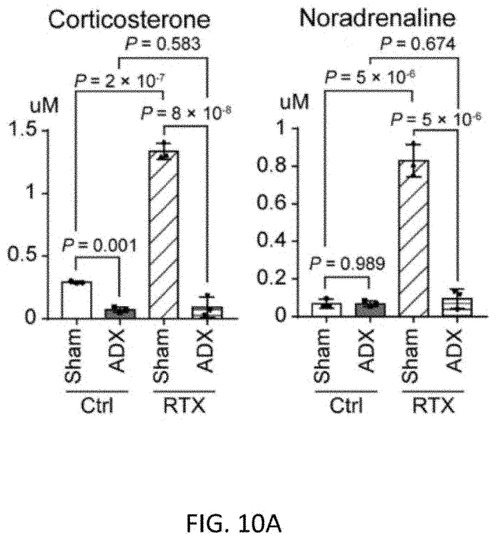

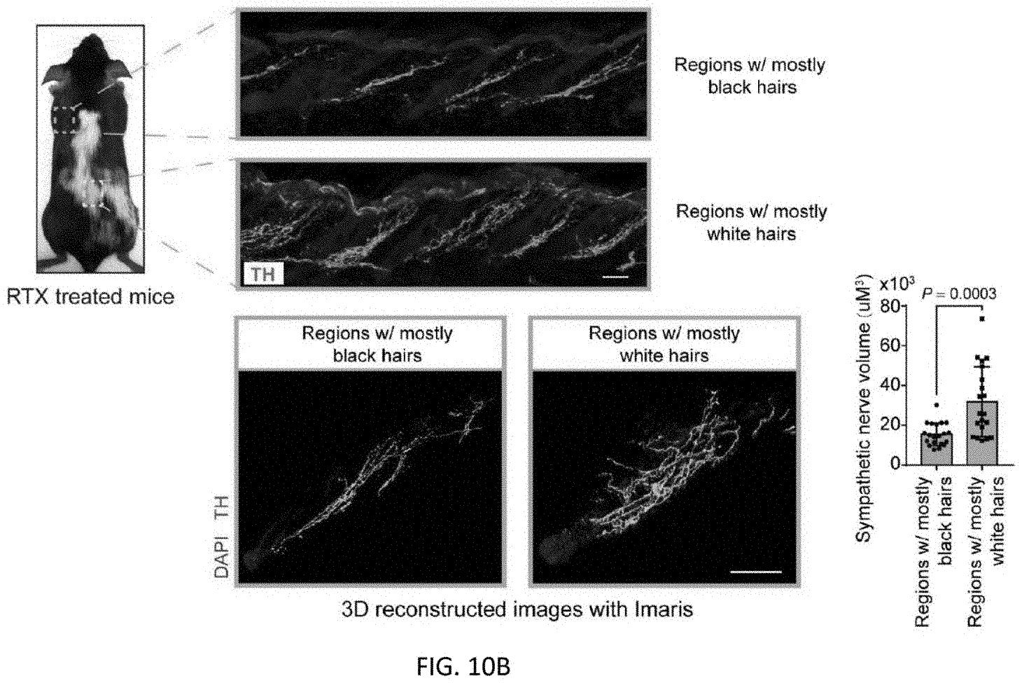

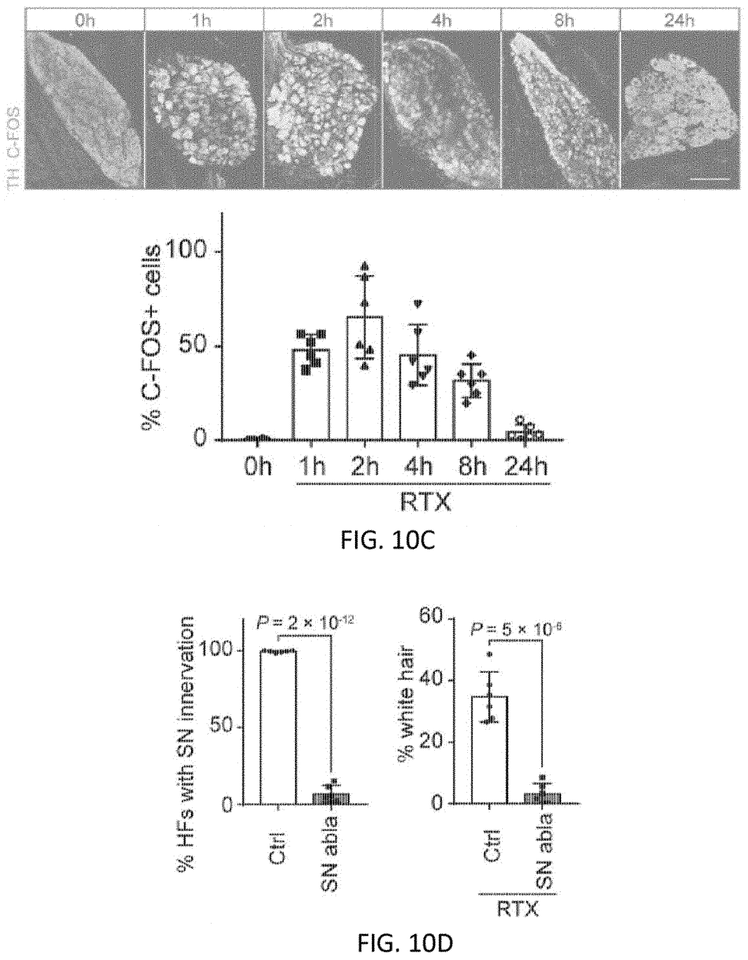

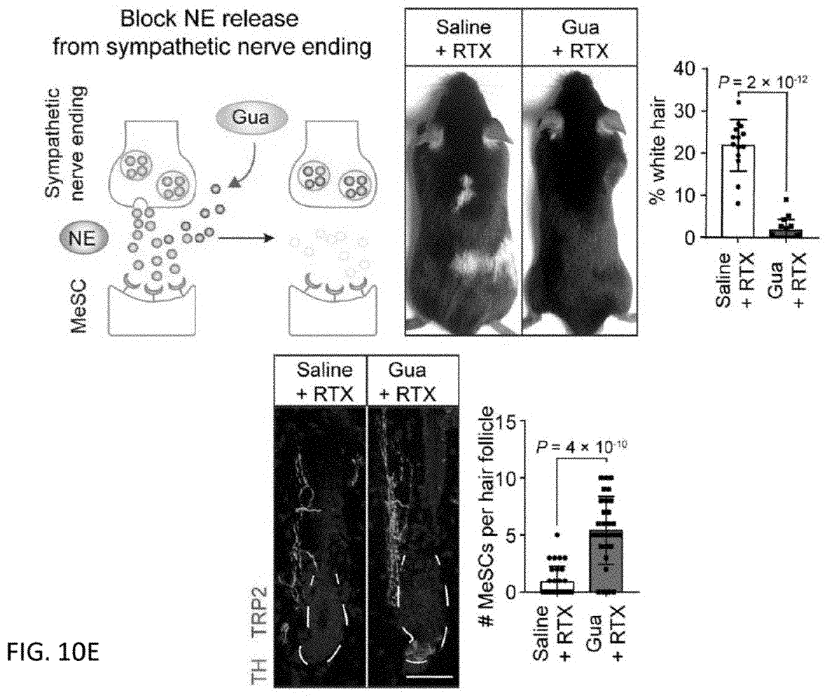

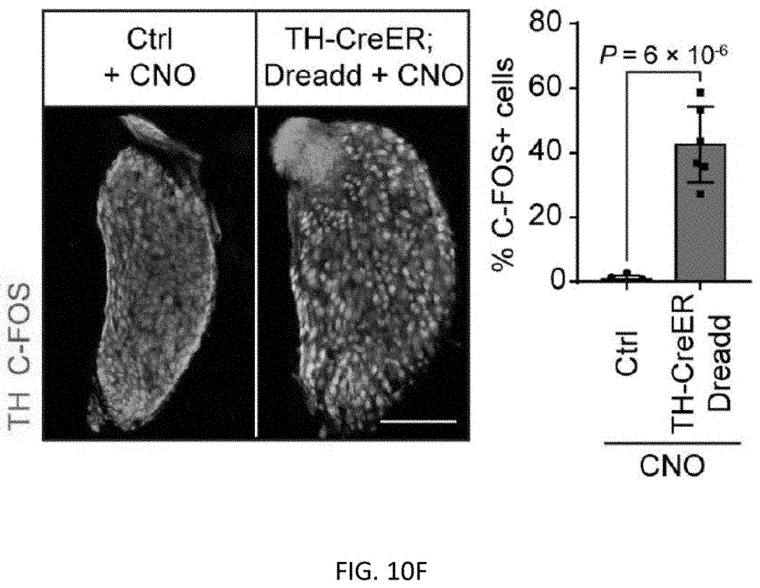

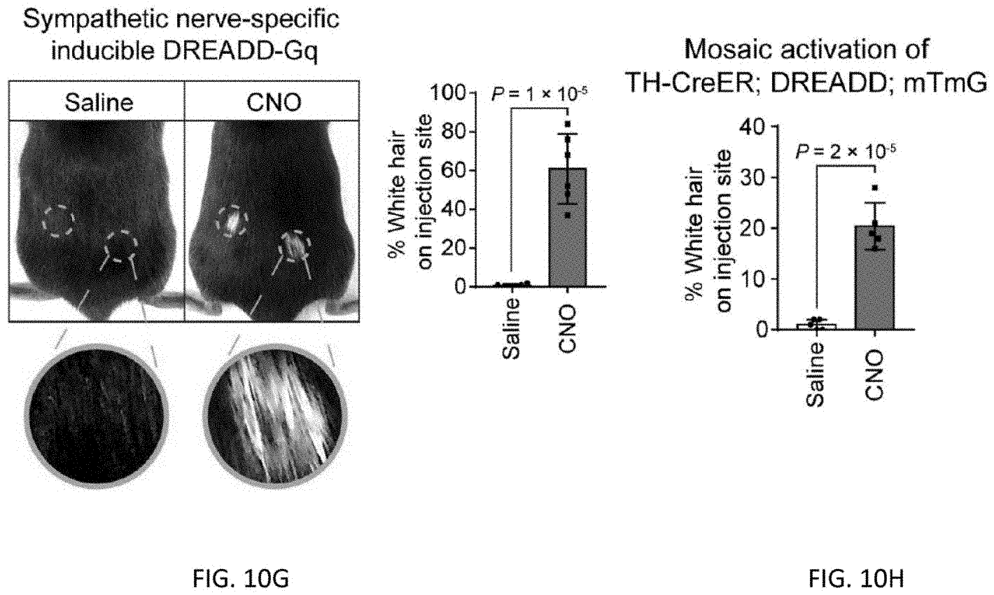

Since the adrenal gland is a major source of norepinephrine under stress, to determine if adrenal gland-derived norepinephrine mediates stress-induced hair greying, both adrenal glands were surgically removed. Adrenalectomy significantly reduced the levels of corticosterone and norepinephrine in the bloodstream of RTX-injected animals (FIG. 10A). Yet, injection of RTX into adrenalectomized mice still caused hair greying, suggesting that RTX-induced hair greying is independent of hormones or catecholamines from the adrenal glands (FIG. 2D).

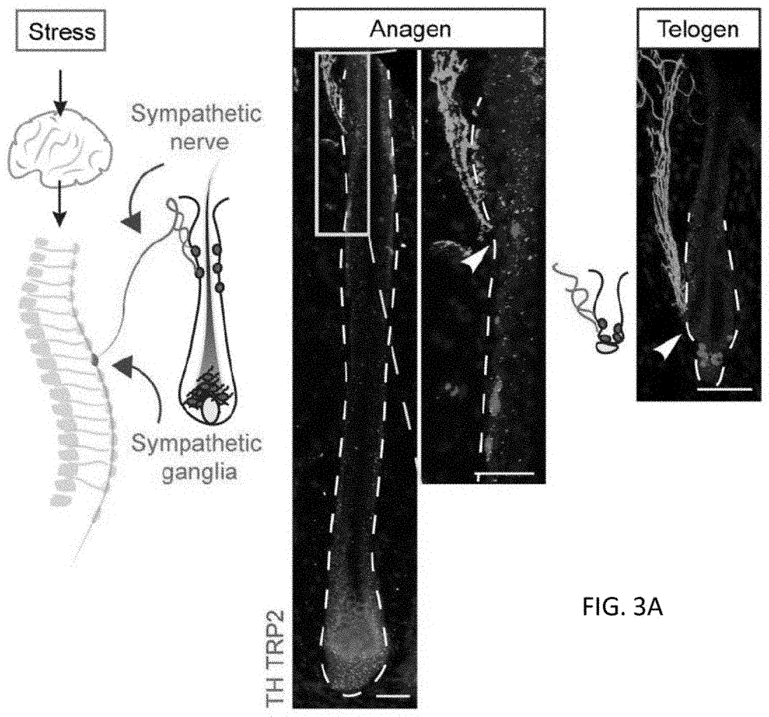

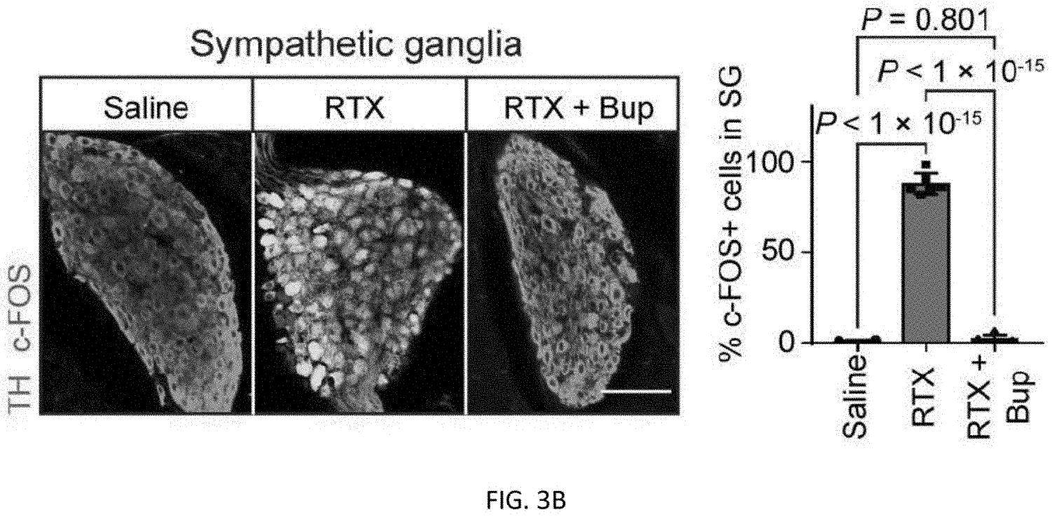

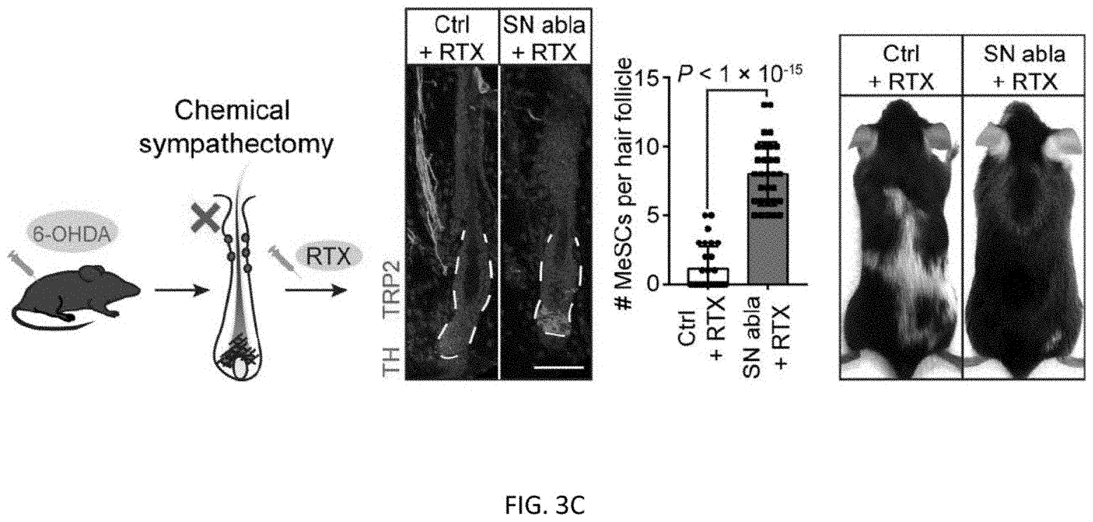

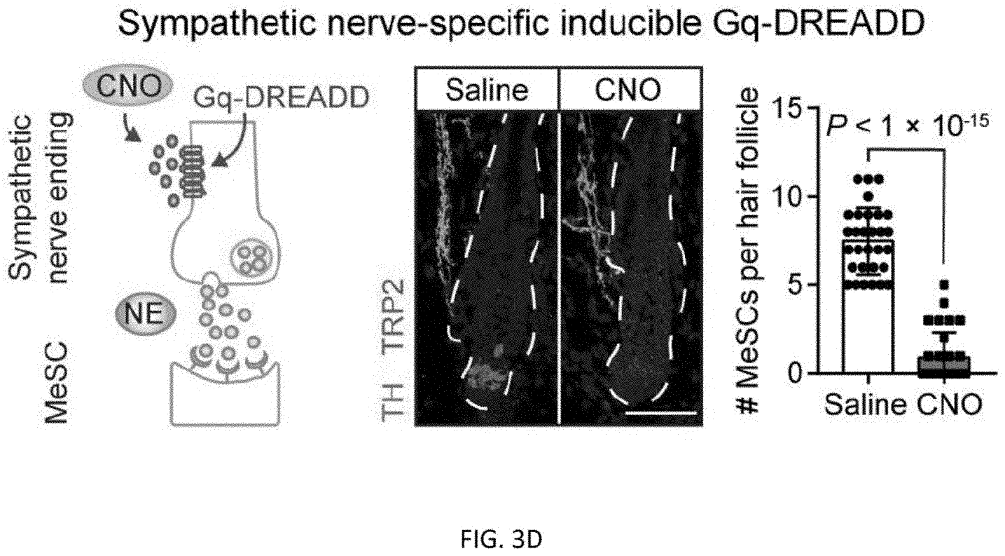

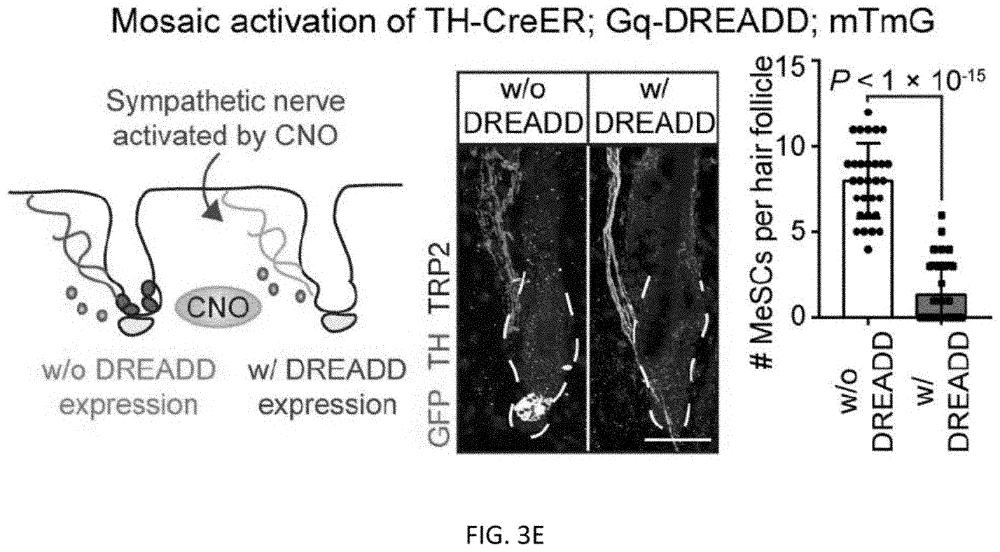

One alternative source of norepinephrine is the sympathetic nervous system. Under stress, the sympathetic nervous system becomes activated to induce fight-or-flight responses through secretion of norepinephrine from peripheral axon terminals [17]. In the skin, sympathetic nerves terminate close to the bulge where MeSCs reside (FIG. 3A). Moreover, skin regions with high numbers of unpigmented hairs also have denser sympathetic innervation (FIG. 10B).

To determine if sympathetic nerves are indeed activated following RTX injection, levels of c-FOS were examined, an immediate early transcription factor reporting neuronal activity [27]. Robust c-FOS induction was detected in the cell bodies of sympathetic neurons within 1 hour after RTX injection, peaking around 2-4 hours, and diminishing after 24 hours, suggesting that RTX injection led to a burst activation of sympathetic neurons (FIG. 3B, FIG. 10C). Moreover, when buprenorphine was injected together with RTX to block pain, sympathetic neurons failed to induce c-FOS (FIG. 3B, right). These data suggest that the sympathetic nervous system becomes highly activated following nociception-induced stress.

To test if activation of sympathetic nerves is responsible for MeSC loss and hair greying under stress, sympathetic nerves were ablated with 6-hydroxy dopamine (6-OHDA), a selective neurotoxin for sympathetic nerves [28]. Sympathectomy blocked RTX-induced hair greying and MeSC loss (FIG. 3C, FIG. 10D), suggesting that sympathetic nerves indeed mediate stress-induced hair greying. In addition, guanethidine, a chemical that blocks norepinephrine release from sympathetic nerve terminals [29], suppressed hair greying and MeSC loss upon RTX injection (FIG. 10E). Collectively, these data suggest that norepinephrine secreted from sympathetic nerve terminals mediates the effect of stress on MeSCs.

To determine if sympathetic nerve activation in the absence of stress is sufficient to drive MeSC loss, a chemogenetic approach was taken using the Designer Receptor Exclusively Activated by Designer Drugs (DREADDs) system [3, 4]. Gq-DREADD is an artificial Gq-protein coupled receptor activated by the inert molecule Clozapine N-Oxide (CNO) but not by endogenous ligands. Activation of Gq-DREADD leads to intracellular calcium release and neuronal firing. TH-CreER; CAG-lsl-Gq-DREADD; Rosa-mT/mG mice were generated, which allowed for artificial activation of sympathetic nerves with CNO (FIG. 3D). Injection of CNO induced c-FOS activation in sympathetic ganglia, confirming the efficacy of this strategy (FIG. 10F). Sympathetic nerve activation with the DREADD system led to loss of MeSCs and hair greying at the sites where CNO was injected (FIG. 3D, FIG. 10G). Moreover, when TH-CreER was activated mosaically by a low dose of tamoxifen, intradermal CNO injection resulted in MeSC loss only in hair follicles innervated by DREADD-positive nerve fibres (recognizable by their membrane GFP expression; FIG. 3E, FIG. 10H). These data suggest that sympathetic nerve activation in the absence of stressors is sufficient to drive MeSC loss. Altogether, the findings suggest that elevated norepinephrine secreted from the sympathetic nerve terminals drives MeSC depletion under stress.

### Stress Drives MeSC Hyper-Proliferation

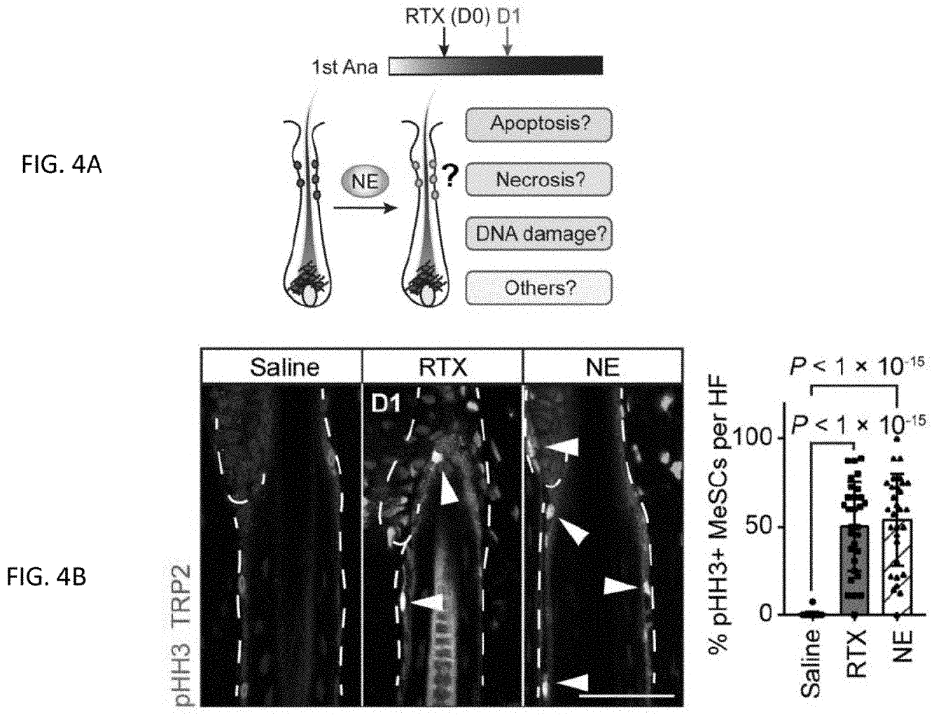

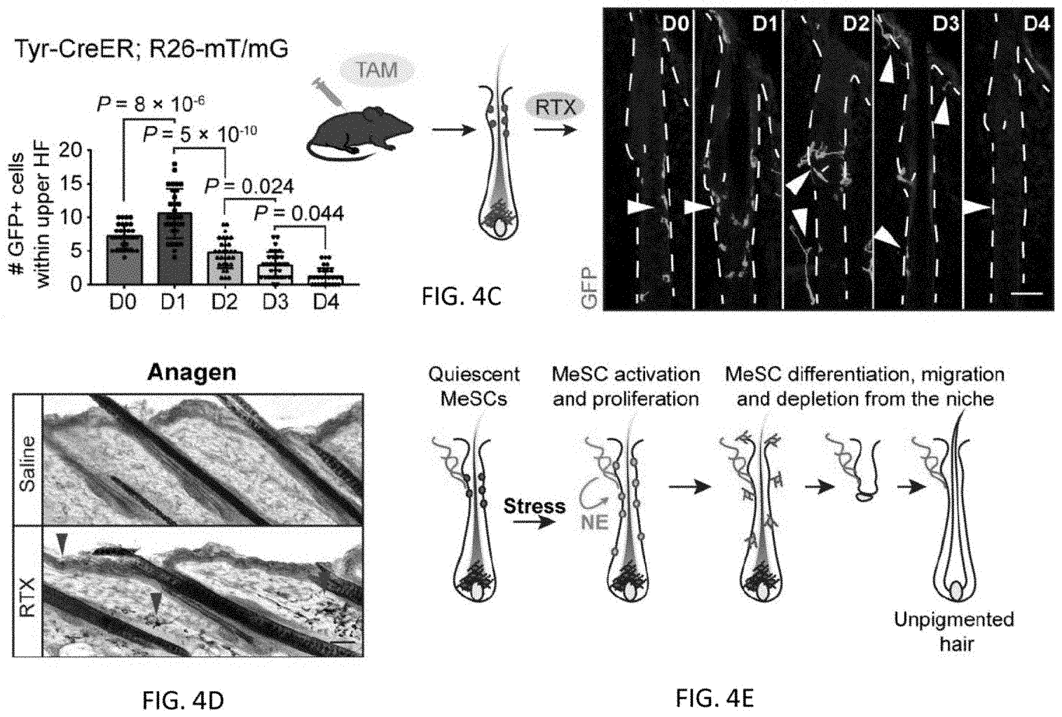

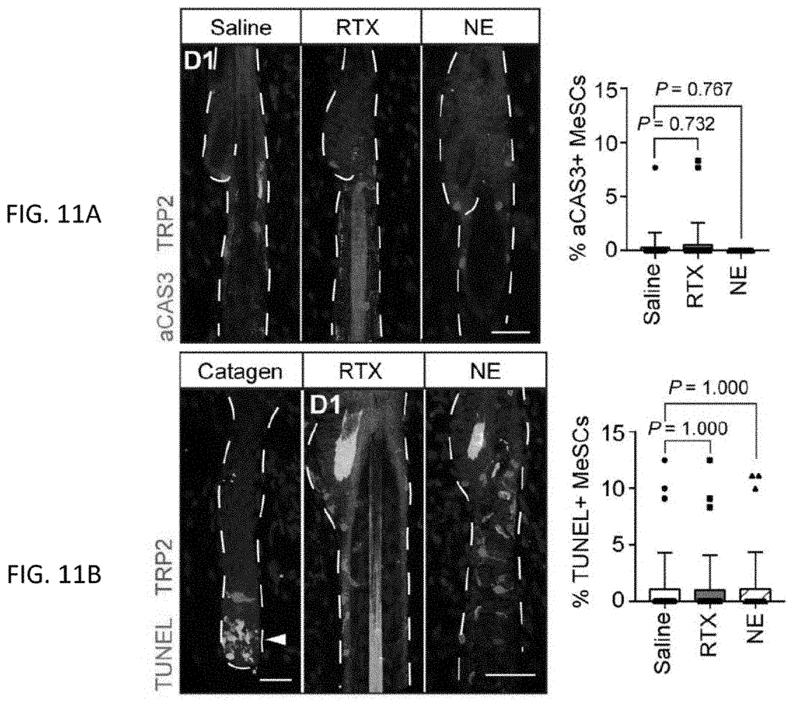

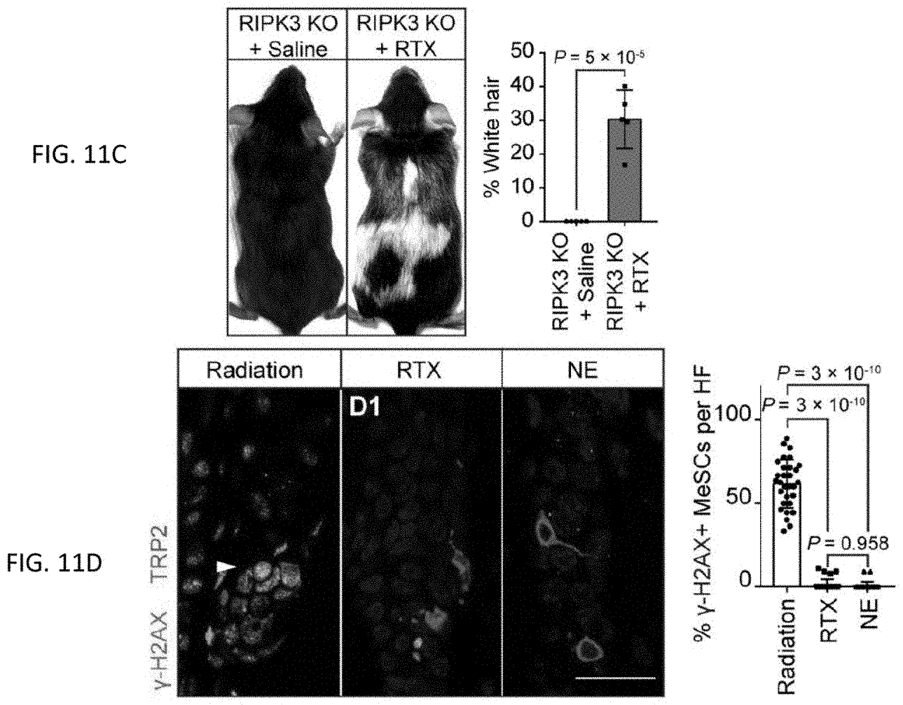

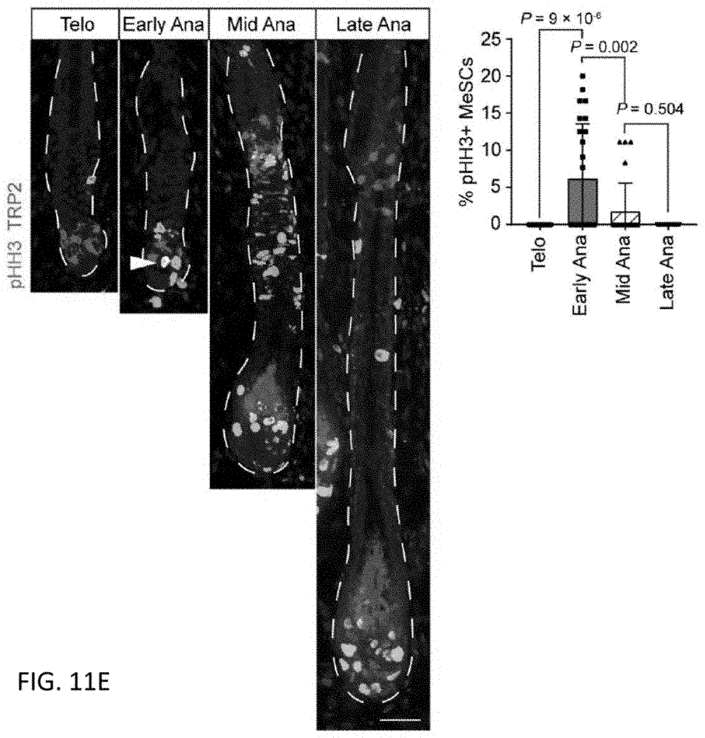

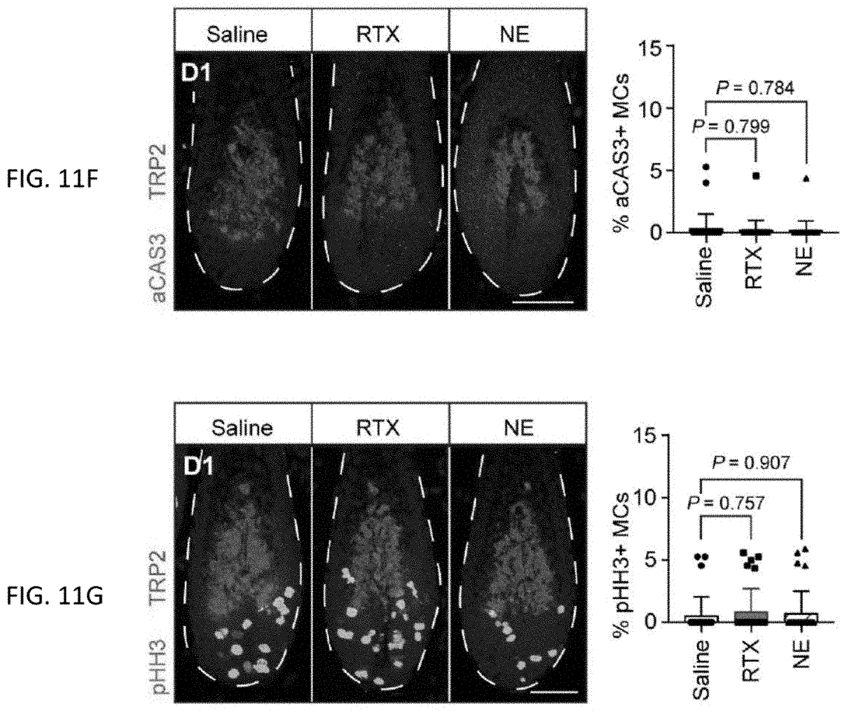

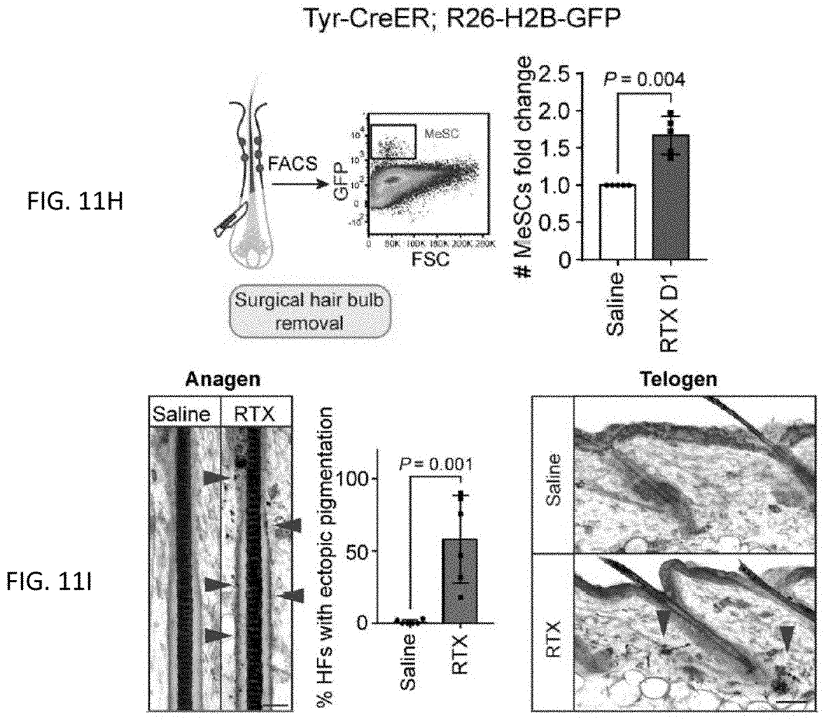

Next, the early changes in MeSCs are aimed to be identified under stress that might account for their loss (FIG. 4A) Immunofluorescence failed to detect active caspase-3 or TUNEL signals in MeSCs before their depletion from the niche upon RTX or norepinephrine injection. Moreover, RTX injection into RIPK3 mutant mice lacking a key kinase for necrosis still caused hair greying (FIGS. 11A-11C). These data suggest stress-induced MeSC loss is not mediated by apoptosis or necrosis. Radiation causes DNA damage in MeSCs, leading to their differentiation within the niche [22]. However, there was a failure to detect gamma-H2AX foci (a hallmark of DNA damage) in MeSCs following RTX or norepinephrine injection, suggesting that stress-induced depletion of MeSCs is not mediated through DNA damage (FIG. 11D).

Quiescence is a key feature of many somatic stem cells [30-33]. Loss of quiescence has been postulated to cause MeSC loss in Bcl2 mutants [10, 34]. To examine if stress alters MeSCs quiescence, RTX or norepinephrine was injected into mice that had entered full anagen, when MeSCs are normally quiescent. A dramatic increase in the number of proliferating MeSCs was seen within 24 hours after RTX or norepinephrine injection—about 50% of MeSCs became positive for Phospho-Histone H3, an M phase marker (FIG. 4B). This number is in sharp contrast to the MeSC proliferation seen in early anagen (˜6%), the only stage when MeSCs proliferate to self-renew (FIG. 11E) [9, 35]. By contrast, no changes in proliferation or apoptosis were observed in mature melanocytes after RTX or norepinephrine injection (FIGS. 11F-11G). These data suggest that elevated norepinephrine forces MeSCs to enter a rapid and abnormally proliferative state, while sparing mature melanocytes.

To monitor changes in MeSCs following stress, Tyr-CreER; Rosa-mT/mG mice were generated, which allowed for the tracing of MeSCs by membrane GFP (FIG. 4C). Consistent with the observation that proliferation is an early response of

MeSCs to stress, a transient increase in GFP positive cells was seen shortly after RTX injection (FIG. 4C, D1, FACS quantified in FIG. 11H). Following this initial phase, many GFP positive cells began to exhibit striking dendritic branching, characteristic of differentiated MeSCs (FIG. 4C, D2). They also began to depart from the bulge—some migrated downwards along the hair follicle, and some migrated out into dermis or epidermis (FIG. 4C, D2 and D3). By Day 3, many GFP positive cells had migrated out of the bulge, and by Day 4, many hair follicles had lost all GFP positive cells in the bulge. Moreover, ectopic pigmentation could be detected along the hair follicle, epidermis, and dermis, places that are normally devoid of pigments (FIG. 4D, FIG. 11I). Collectively, these data suggest that after stress, MeSCs undergo rapid proliferation followed by differentiation and migration, leading to their loss from the niche (FIG. 4E).

### Transcriptome Analyses of MeSCs

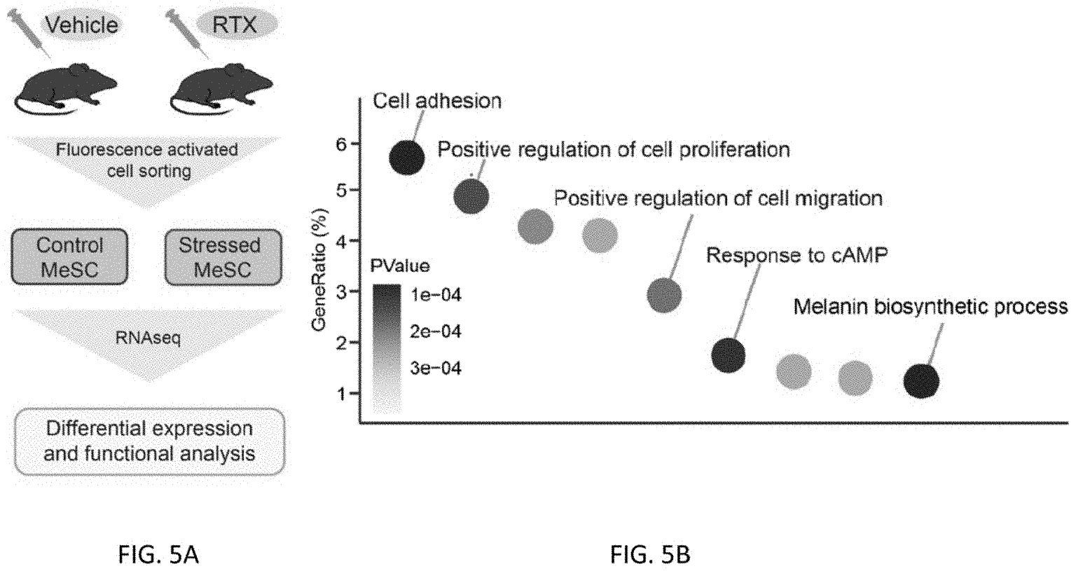

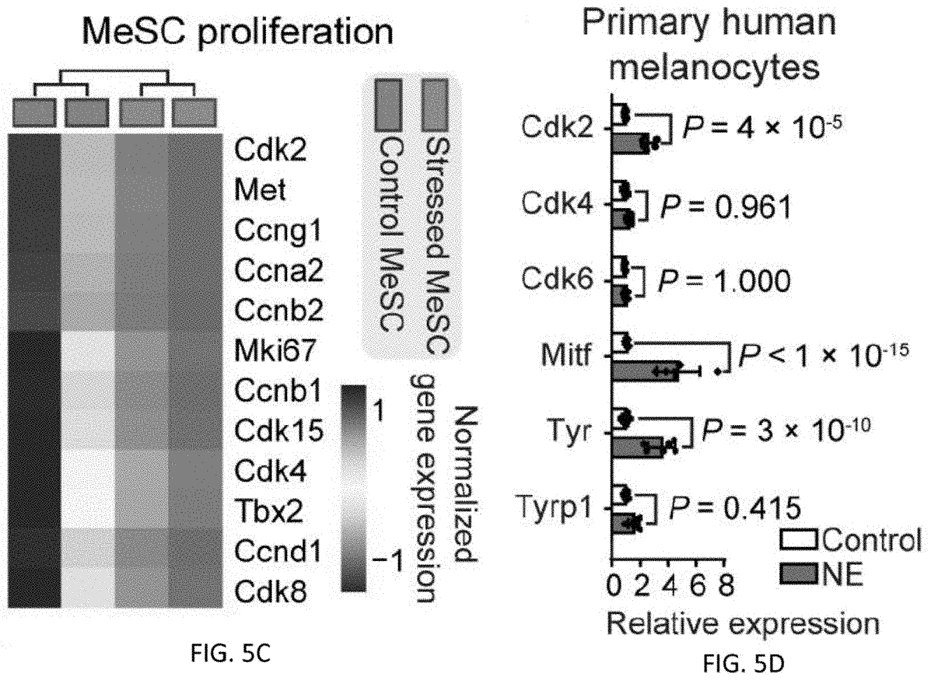

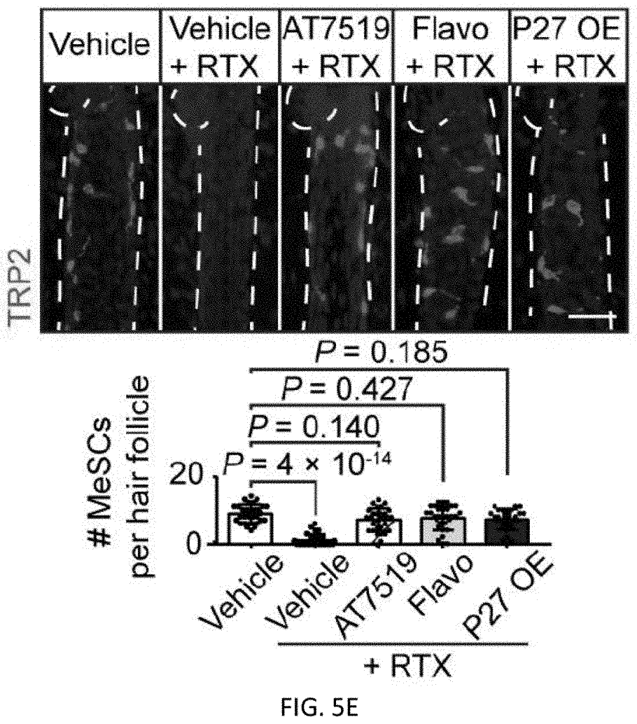

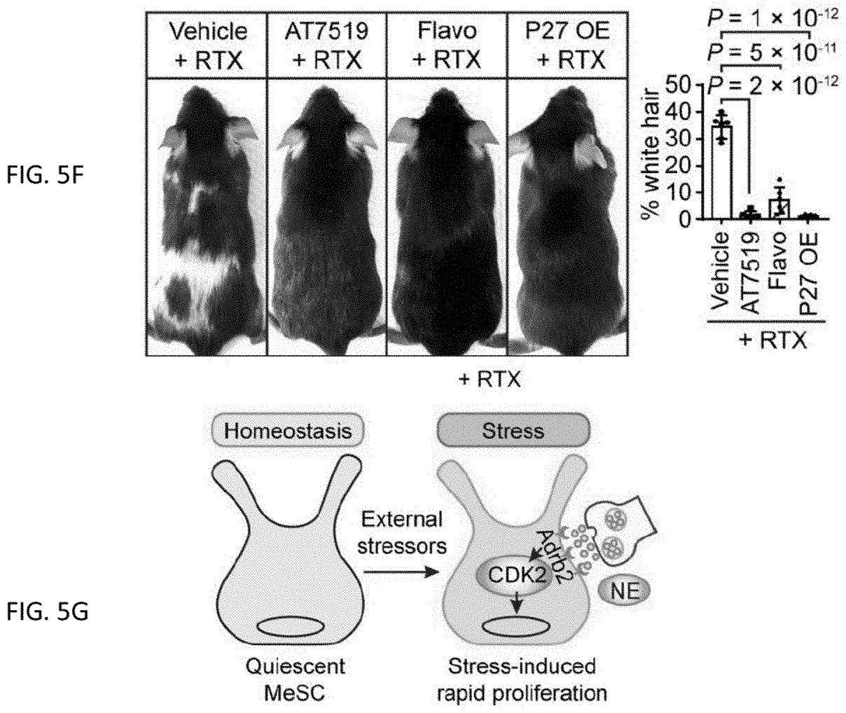

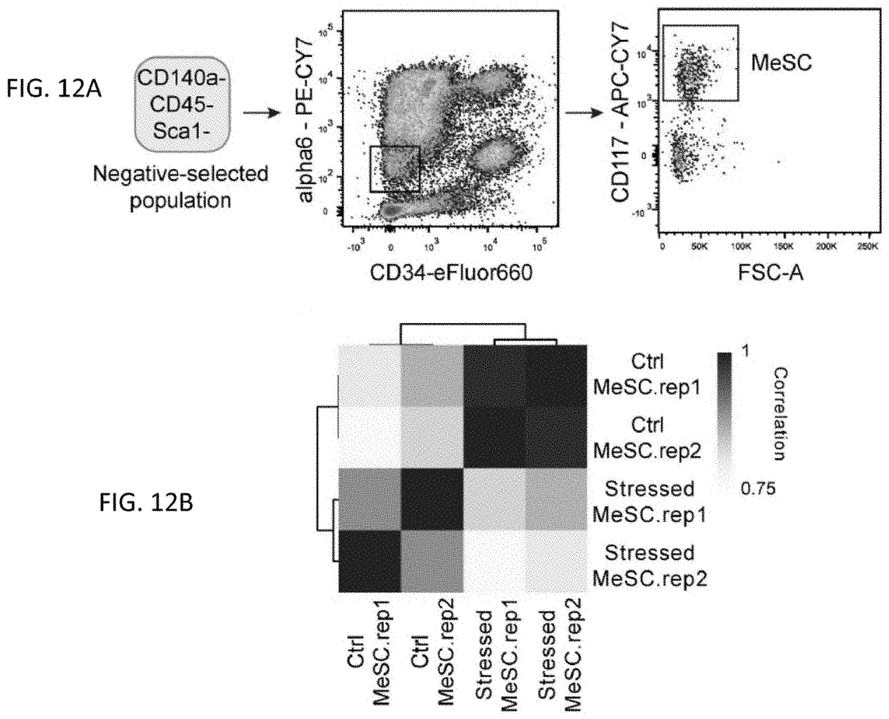

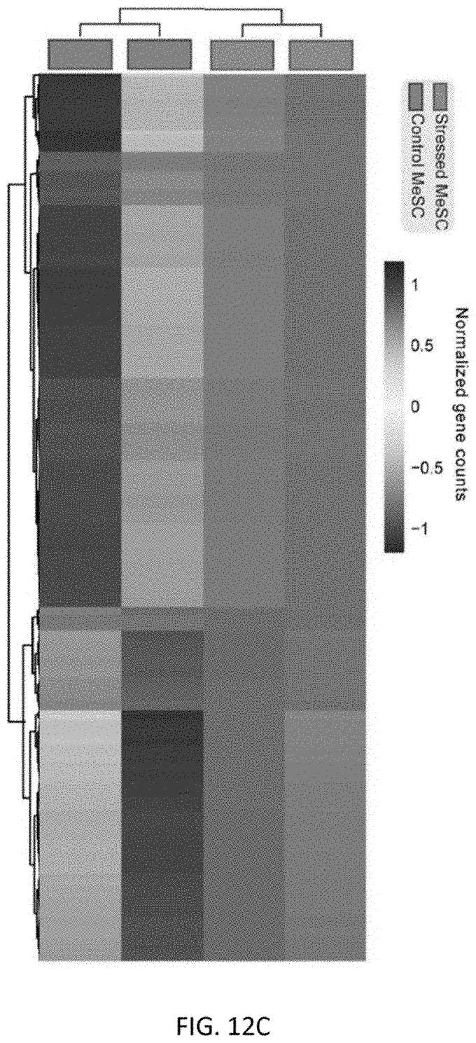

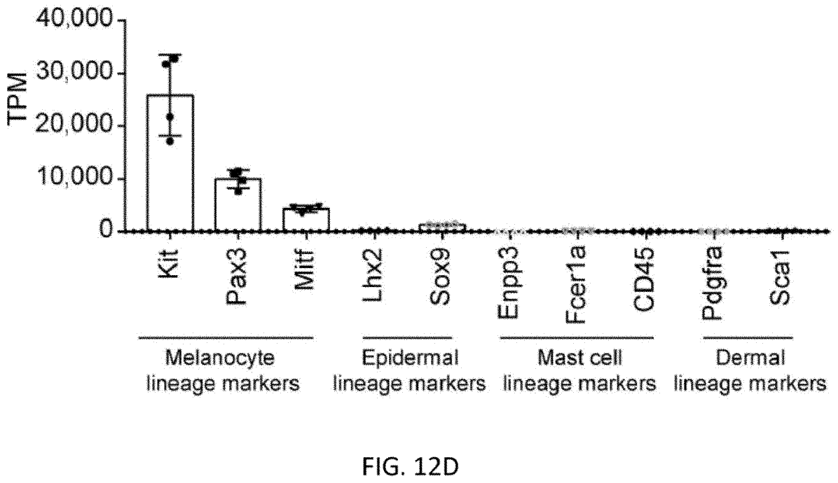

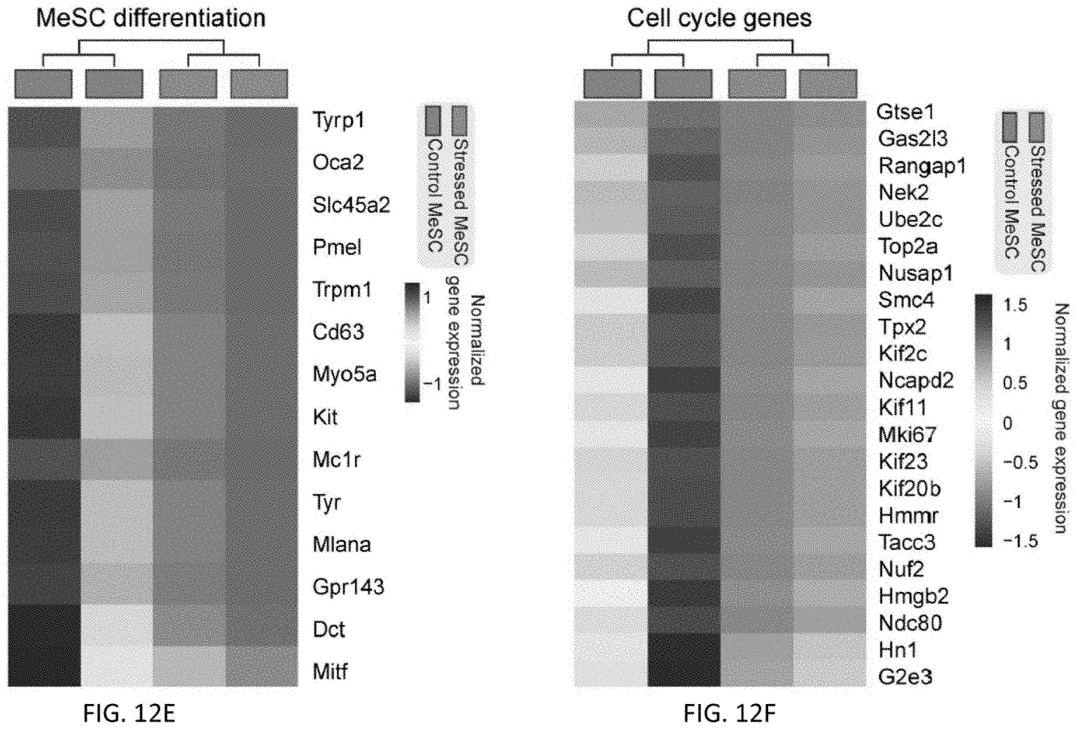

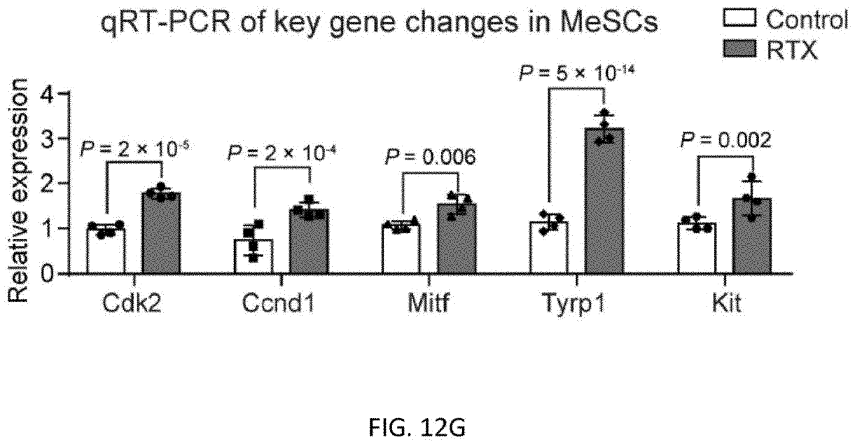

To discover the molecular mechanisms driving MeSC loss under stress, RNA-seq was conducted using FACS-purified MeSCs from control and RTX-treated animals 12 hours after RTX injection, before MeSCs showed phenotypic differences (FIG. 5A, FIGS. 12A-12C). Examination of marker gene expression for diverse skin cell types confirmed successful enrichment for MeSCs (FIG. 12D). To uncover major molecular changes, Gene Ontology (GO) enrichment analysis was conducted (FIG. 5B). A list of known genes associated with MeSC proliferation and differentiation was curated (FIG. 5C, FIG. 12E). Moreover, a list of genes previously denoted for cell cycle entry was utilized to assess if cell cycle regulators are altered at the transcriptional level (FIG. 12F) [36]. Some of these key changes were also verified by quantitative RT-PCR (qRT-PCR) (FIG. 12G). Collectively, changes in several cell cycle regulators were identified in stressed MeSCs, including Cyclin-dependent kinase 2 (Cdk2), a key promoter of G1 to S transition. Receptors for ligands that promote MeSC proliferation, differentiation, and migration, including c-Kit [37] and Mc1r [38], were also upregulated. In addition, genes involved in melanogenesis [19], including Mitf, Tyrpl , Tyr, Oca2, and Pme1, were upregulated (FIG. 5C, FIGS. 12E and 12G). These data suggest that MeSCs upregulate proliferation and differentiation programs following stress. Furthermore, norepinephrine exposure also led to a rapid induction of proliferation genes like Cdk2, and differentiation genes like Mitf and Tyr in cultured human melanocyte cells (FIG. 5D). These data suggest that norepinephrine elicits similar responses in both human and mouse melanocyte lineages.

### Blocking Proliferation Preserves MeSCs

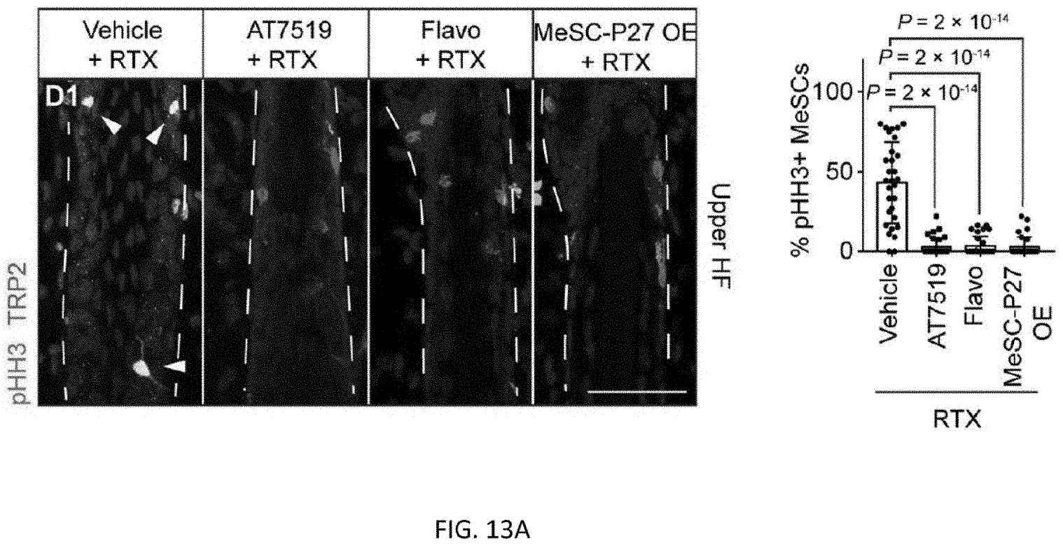

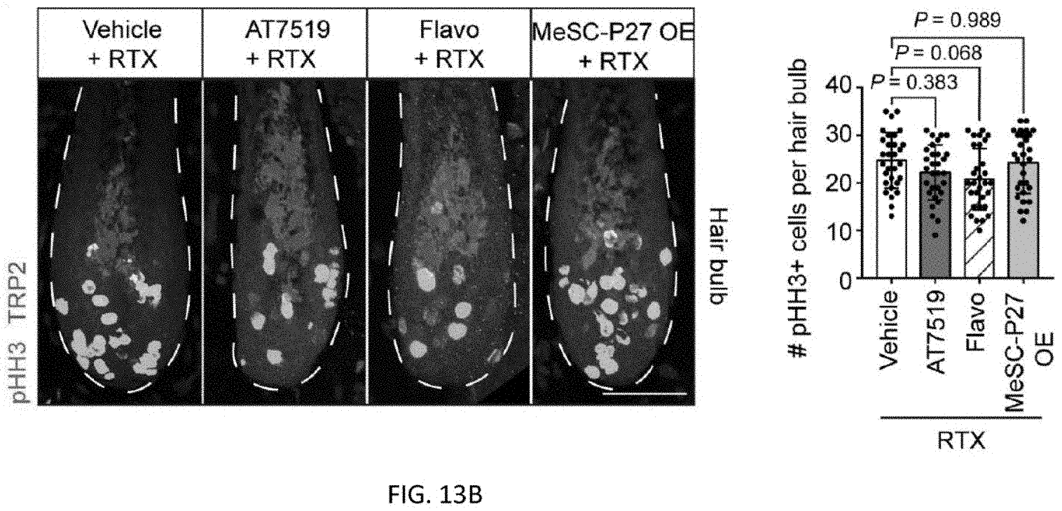

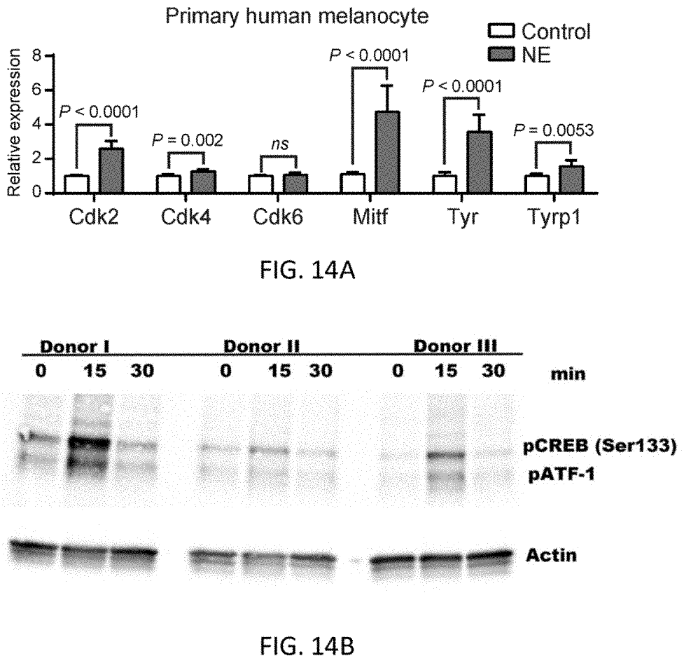

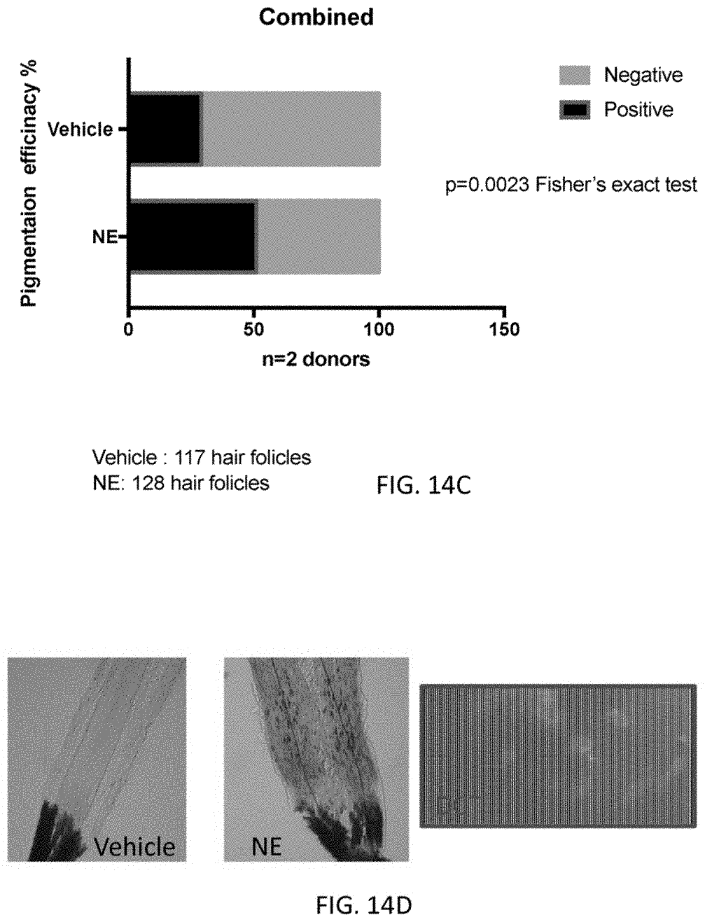

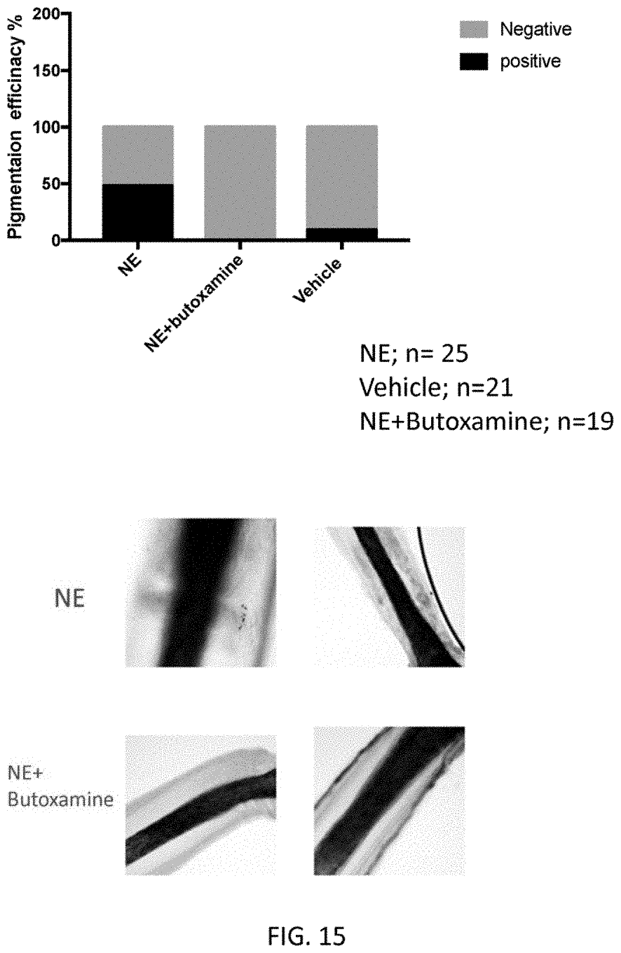

Since MeSCs first lose quiescence upon stress, it was asked if transient suppression of proliferation early in the stress response might prevent their depletion. For this, RTX was injected at full anagen, and applied CDK inhibitors (AT7519 or Flavopiridol) topically to suppress proliferation transiently until 48 hours post injection [39, 40]. MeSCs in RTX-injected animals treated with CDK inhibitors remained quiescent and were preserved in the niche (FIG. 5E, FIG. 13A). Proliferation of cells in the hair bulb remained largely normal, likely because the penetration of inhibitors into subcutaneous regions in full anagen was limited (FIG. 13B). To further establish that MeSC loss can be prevented by inhibiting MeSC proliferation, a genetic model was generated (Tyr-CreER; Rosa-lsl-rtTA; TetO-P27) in which the CDK inhibitor P27 can be transiently induced specifically in MeSCs with doxycycline. Induction of P27 expression in MeSCs alone suppressed aberrant MeSC proliferation and preserved MeSCs in the niche under stress (FIG. 5E, FIG. 13A). These preserved MeSCs displayed an undifferentiated morphology and retained functionalities, as newly regenerated hairs in subsequent cycles maintained pigmentation (FIG. 5F). Collectively, these data suggest that loss of quiescence drives MeSC depletion in stress, and that suppression of MeSC proliferation is sufficient to prevent their loss.

### Discussion

Acute stress is known to cause transient and beneficial “fight-or-flight” responses essential for survival. Here, it is demonstrated that acute stress can also cause non-reversible depletion of somatic stem cells through activation of the sympathetic nervous system, resulting in permanent damage to tissue regeneration (FIG. 5G). The findings support the emerging notion that the sympathetic nervous system not only regulates body physiology, but also influences diverse processes in development and tissue maintenance [13, 41-43]. The adrenal glands are the central regulators of stress responses. However, it is shown that the adrenal gland-derived circulating stress hormones and catecholamines do not drive changes in MeSCs under stress. Since sympathetic nerves innervate essentially all organs, acute stress might have a broad and rapid impact on many tissues via neuronal signals rather than circulating hormones.

Why does such a nerve-stem cell interaction exist? The connection between the nervous system and pigment-producing cells is likely conserved during evolution. Cephalopods like squid, octopus, or cuttlefish have sophisticated colouration systems that allow them to change colour for camouflage or communication. Neuronal activities control their pigment-producing cells (chromatophores), allowing rapid changes in color in response to predators or threats [44]. Therefore, an attractive hypothesis is that sympathetic nerves might modulate MeSC activity, melanocyte migration, or pigment production in situations independent of the hair cycle—for example, under bright sunlight or UV irradiation [45]. Under extreme stress, however, hyperactivation of neuronal activities over-stimulates the pathway, driving MeSC depletion.

MeSCs also exhibit ectopic differentiation and depletion with age [10, 20]. Of relevance, patients who have undergone partial sympathectomy develop fewer numbers of unpigmented hairs on the sympathectomized side with age [46, 47].

### Methods

### Animals

C57BL/6J, Tyr-CreER, K15-CrePGR, Rag1 mutant, CD11b-DTR, GR flox, CAG-lsl-Gq-DREADD, Rosa-H2BGFP/mCherry, Rosa26-mT/mG, Rosa-lsl-rtTA, and RIPK3 mutant mice were obtained from the Jackson Laboratory. adrb2 flox [48] mice were originally generated by Dr. Gerard Karsenty (Columbia University) and provided to us by Dr. Paul Frenette (Albert Einstein College of Medicine). TH-CreER [49] mice were generated and provided by Dr. David Ginty (Harvard Medical School). TetO-P27 [50] mice were originally generated by Dr. Gillian K. Cady (Roswell Park Cancer Institute) and provided to us by Dr. Valentina Greco (Yale School of Medicine). All experiments used balanced groups of male and female mice. All experiments are conducted and compared using mice of the same hair cycle stage in comparable age range (P20-P25 for 1^{st} telogen, P31-P36 for full anagen, and P50-P60 for 2^{nd} telogen, or long-term monitoring as specified). To monitor hair cycle, mice were shaved at weaning to monitor skin colour changes and confirmed by skin sections. The acquisition of human melanocyte cells was carried out in compliance with the IRB policies at MGH. All animals were maintained in an Association for Assessment and Accreditation of Laboratory Animal Care-approved animal facility at Harvard University, Harvard Medical School, and Ribeirao Preto Medical School. Procedures were approved by the Institutional Animal Care and Use Committee of all institutions and were in compliance with all relevant ethical regulations.

### Stress Procedures

Restraint and chronic unpredictable stress (CUS) procedures were performed as previously described [11-14]. Briefly, for restraint stress, C57BL/6J mice were kept in a restrainer (Fisher Scientific 12972590) for 4 hours a day for five days starting from mid-anagen (P28-P30). Hairs were depilated to induce hair regeneration when their hair cycle reached telogen. Mice were depilated 4 times in total to monitor long-term changes. For CUS, C57BL/6J mice were exposed to a combination of stressors. Two of the stressors were applied each day. The stressors include cage tilt, isolation, damp bedding, rapid light/dark changes, overnight illumination, restraint, empty cage, and 3× cage change. All stressors were randomly repeated in consecutive weeks.

### Drug Treatments

For RTX injection, mice received injections of RTX (30-100 μg/kg) in the flank for 1-3 days as described previously [15, 16, 51-56]. RTX was prepared in 2% DMSO with 0.15% Tween 80 in PBS. Control mice were treated with the vehicle only. RTX injection was done either in full anagen (P31-P36) or in 1^{st} telogen (P21). For corticosterone feeding, 35 μg/ml corticosterone (Millipore Sigma, C2505) was dissolved in 0.45% hydroxypropyl-β-cyclodextrin and provided in drinking water. Mice were treated for three days (P28-P30). Control mice received the vehicle water (0.45% β-cyclodextrin). For analgesia, mice were injected with buprenorphine (0.1 mg/kg) 4 hours before RTX injection, and every 6 hours after RTX injection for 2 days. For tamoxifen treatment, tamoxifen was diluted in corn oil to a final concentration of 20 mg/ml. To induce recombination, 20 mg/kg was injected intraperitoneally once per day for 4-7 days. For mosaic induction of Tyr-CreER and TH-CreER, 20 mg/kg tamoxifen was injected intraperitoneally once per day for 3 days. For intradermal norepinephrine injection, norepinephrine (Sigma-Aldrich 489350) solution was prepared freshly by dissolving in 0.1% ascorbic acid in 0.9% sterile NaCl to a final concentration of 2 mM. 50 μm was injected intradermally into experimental animals together with fluorescent beads at full anagen (P31˜P36). Control animals were injected with equivalent volume of vehicle (0.1% ascorbic acid in 0.9% sterile NaCl) with fluorescent beads. The injection sites were marked using water resistant ink. For sympathetic nerve ablation, 6-hydroxydopamine hydrobromide (6-OHDA, Sigma 162957) solution was prepared freshly by dissolving 6-OHDA in 0.1% ascorbic acid in 0.9% sterile NaCl. 100 mg/kg (body weight) of 6-OHDA was injected intraperitoneally daily from P18 to P22. Control animals were injected with equivalent volume of vehicle (0.1% ascorbic acid in 0.9% sterile NaCl). Ablation efficiency in the skin was confirmed by immunofluorescence staining For guanethidine treatment, mice were intraperitoneally injected with 30 mg/kg (body weight) of guanethidine (Sigma-Aldrich, 1301801), once a day for 3 consecutive days prior to RTX administration at full anagen (P31˜P36). For Induction of Gq-DREADD, 50 μl CNO (1 mg/ml in 0.9% sterile saline) was injected intradermally into experimental animals together with fluorescent beads at full anagen (P31˜P36). Control animals were injected with equivalent volume of vehicle (0.9% sterile saline) together with fluorescent beads. For diphtheria toxin administration, diphtheria toxin (DT, Sigma-Aldrich) was dissolved in 0.9% saline (0.1 mg/ml). For ablation, CD11b-DTR transgenic mice were intraperitoneally injected with 25 ng/g (body weight) DT daily 3 days before RTX injection at full anagen (P31˜P36). 20 ng/g (body weight) DT was injected every three days after RTX injection until harvesting. For inhibitor treatment, mice were shaved and pre-treated with 5 mg/kg (body weight) AT7519 (Cayman Chemical 16231) or Flavopiridol (Cayman Chemical 10009197) in ethanol topically 48 hours and 24 hours before RTX injection, at the time of RTX injection, and 24 hours and 48 hours after injection. For P27 expression induction, mice were fed with Doxycycline Rodent Diet (VWR 89067-462) for three days before the RTX treatment and three days after. RTX was given at Anagen VI (P31˜P36).

### Quantification of Unpigmented Hairs

For Restraint and CUS, unpigmented hairs were quantified by plucking ˜100 hairs from 3-4 regions of the skin across the anterior to posterior end, and the percentage of white hairs were calculated by dividing the number of white hairs by the total number of hairs plucked. For RTX injection experiments, the percentage of white hair regions was calculated by dividing the size of white hair areas with the size of the whole skin (both areas were measured using ImageJ). For intradermal injection experiments (NE or CNO), unpigmented hairs were quantified by plucking ˜100 hairs from each injection site (marked by water resistant ink at the time of injection), and the percentage of white hairs were calculated by dividing the number of white hairs by the total number of hairs plucked.

### Histology and Immunohistochemistry

Mouse skin samples were fixed using 4% paraformaldehyde (PFA) for 15 minutes at room temperature, washed 6 times with PBS, and immersed in 30% sucrose overnight at 4° C. Samples were then embedded in OCT (Sakura Finetek). 35˜50 μm sections were fixed in 4% paraformaldehyde (PFA) for 2 minutes and washed with PBS and PBST. Slides were then blocked using blocking buffer (5% donkey serum; 1% BSA, 2% cold water fish gelatin in 0.3% Triton in PBS) for 1 hour at room temperature, followed by staining with primary antibodies overnight at 4° C. and secondary antibody for 4 hours at room temperature. For sympathetic nerve density quantification, 90 μm sections were used. EdU was developed for 1 hour using the Click-It reaction according to manufacturer instructions (Thermo Fisher Scientific). TUNEL assay was performed according to manufacturer instructions (Roche). Fontana—Masson staining was performed according to manufacturer instructions (Market Lab ML7255). Antibodies used: TRP2 (Santa Cruz 10451, 1:800), tyrosine hydroxylase (rabbit, Millipore AB152, 1:1000 or sheep, Millipore AB1542, 1:150-1:300), c-Fos (Abcam, 190289, 1:1000), γ-H2AX (Cell Signaling, 9718, 1:400), phospho-histone H3 (rabbit, Cell Signaling Technology 3377S, 1:500), cleaved caspase 3 (rabbit, Cell Signaling Technology 9664S, 1:400), GFP (rabbit, Abcam ab290, 1:1000 or chicken, Ayes labs GFP-1010, 1:200), CD3 (eBioscience 14-0032-81, 1:800), CD11b (eBioscience 14-0112-81, 1:800), Phospho-CREB (Cell Signaling 9198, 1:800), MITF (Abcam ab12039, 1:400).

### Measurement of Stress Hormones

50 μl of blood plasma was collected from each mouse and transferred into a 1.5 ml microcentrifuge tube. 10 μl of internal solution was added to each sample followed by 100 μl of water and 640 μl of methanol. Samples were incubated at −20° C. for 1 hour, then centrifuged 30 minutes at maximum speed at −9° C. The supernatant was transferred to a new tube and dried under N2 flow and resuspended in 50 μl methanol and transferred to micro-inserts. All samples were run on an Agilent 6460 Triple Quad LC/MS with an Agilent 1290 Infinity HPLC. For corticosterone-treated mice, plasma corticosterone levels were determined by ELISA according to the manufacturer's instruction (Arbor Assays, K014-H1).

### Radiation

Ten-week-old C57BL/6J mice were gamma irradiated (137-Cs source) with a split 10.5 grey split dose. Mice were transplanted with 300,000 whole bone marrow cells to ensure survival after lethal irradiation.

### FACS

Mouse dorsal skin was collected, and the fat layer was removed by gentle scrapping from the dermal side. The skin was incubated in 0.25% collagenase in HBSS at 37° C. for 35-45 minutes on an orbital shaker. Single cell suspension was collected by gentle scraping of the dermal side and filtering through 70 μm and 40 μm filters. The epidermal layer was incubated in trypsin-EDTA at 37° C. for 35-45 minutes on an orbital shaker. Single cell suspension was collected by gentle scraping of the epidermal side and filtering through 70 mn and 40 μm filters. The single cell suspension was centrifuged for 5 minutes at 4° C., resuspended in 0.25% PBS in PBS, and stained with fluorescent dye-conjugated antibodies for 30 minutes. For late anagen skin samples, the bottom parts of the hair follicles containing mature melanocytes were removed by gentle scrapping under dissection microscope. The MeSCs located close to the bulge remained and were verified by immunostaining. Antibodies used: CD140a (Invitrogen 13-1401-82, 1:200), CD45 (Invitrogen 13-0451-82, 1:400), Sca1 (Invitrogen 13-5981-82, 1:1000), CD34 (Invitrogen 13-0341-82, 1:100), CD117 (Biolegend 135136, Dilution 1:400). See a protocol published at protocol exchange website for a step-to-step instruction [57].

### RNA Isolation

RNA was isolated using a RNeasy Micro Kit (Qiagen), using QIAcube according to manufacturer instructions. RNA concentration and RNA integrity were determined by Bioanalyzer (Agilent, Santa Clara, Calif.) using the RNA 6000 Nano chip. High quality RNA samples with RNA Integrity Number ≥8 were used as input for RT-PCR and RNA-sequencing.

### Cell Culture

Primary human melanocytes were derived from neonatal foreskin as previously described [58] and cultured in Medium 254 (Invitrogen, Thermo Fisher Scientific). Melanocytes (passages 2 and 4) were starved for 24 hours in HAM's F-10 +1% penicillin/streptomycin/glutamine before adding NE (10 uM).

### Quantitative Real-Time PCR

The cDNA libraries were synthesized using Superscript IV VILO master mix with ezDNase (Thermo Fisher). Quantitative real time PCR was performed using power SYBR green (Thermo Fisher) in an ABI QuantStudio6 Flex qPCR instrument. Ct values were normalized to an internal control of beta-actin.

### Imaging and Image Analysis

All images were acquired using a Zeiss LSM 880 confocal microscope or Keyence microscope using ×20 or ×40 magnification lenses. Images are presented as Maximum Intensity Projection images. For colocalization analysis, images are presented as a single Z stack. For sympathetic nerve density quantification, TH staining of sympathetic nerves was performed on 90 uM thick skin section samples to ensure the capture of all fibres innervating each hair follicle. Sympathetic nerves innervating individual hair follicles were selected and imaged using a Zeiss LSM 880 confocal microscope. 3D surfaces of TH staining were created using Imaris x64 9.3.0 software and the volume was measured and compared. To quantify cell numbers (MeSC numbers, cell death events, proliferation events) within a hair follicle, immunofluorescence staining images of skin sections from multiple regions across the body were used. The number of cells were counted manually or by using ImageJ.

### Statistical Analysis

Statistical analyses were performed with Prism 7.00 using unpaired two-tailed Student's t-test, One-Way or Two-Way ANOVA. All statistical tests performed are indicated in the figure legends. The data are presented as mean ±SD.

### RNA-Sseq and Computational Analysis

MeSCs were purified using FACS from control and stressed mice skin samples at telogen based on their expression of CD117 [7], starting from a population that is negative for CD140a, CD45, Sca1, and CD34 [57]. 2 ng of total RNA from each sample were used to generate RNA-seq libraries using a SMART-Seq v4 Ultra Low Input RNA kit (Takara, 634888) and Nextera XT DNA Library Preparation Kit (Illumina, FC-131-1024). Single-end sequencing reads were obtained using Illumina NextSeq 500 platform. Sequencing reads from RNA-seq libraries were trimmed using Trim Galore! (bioinformatics.babraham.ac.uk/projects/trim_galore/) and aligned to the mouse reference genome (mm10) using STAR aligner [59]. Reads with alignment quality <Q30 were discarded. Gene expression levels were normalized and differential genes were calculated using DEseq2 package in R [60]. Gene set functional enrichment analysis was performed using David [61, 62]. Transcripts Per Kilobase Million (TPM) calculated from count tables of Control MeSC samples were used to determine the expression levels of adrenergic receptors and glucocorticoid receptor shown in FIG. 2C.

REFERENCES

1. Ephraim, A. J. On sudden or rapid whitening of the hair. AMA Arch Derm 79, 228-236 (1959).

2. Navarini, A. A. & Nobbe, S. Marie Antoinette syndrome. Arch Dermatol 145, 656-656 (2009).

3. Alexander, G. M. et al. Remote control of neuronal activity in transgenic mice expressing evolved G protein-coupled receptors. Neuron 63, 27-39 (2009).

4. Zhu, H. et al. Cre-dependent DREADD (designer receptors exclusively activated by designer drugs) mice. genesis 54, 439-446 (2016).

5. Milller-Rover, S. et al. A comprehensive guide for the accurate classification of murine hair follicles in distinct hair cycle stages. Journal of Investigative Dermatology 117, 3-15 (2001).

6. Hsu, Y.-C., Li, L. & Fuchs, E. Emerging interactions between skin stem cells and their niches. Nature Medicine 20, 847-856 (2014).

7. Chang, C.-Y. et al. NFIB is a governor of epithelial-melanocyte stem cell behaviour in a shared niche. Nature 495, 98-102 (2013).

8. Rabbani, P. et al. Coordinated activation of Wnt in epithelial and melanocyte stem cells initiates pigmented hair regeneration. Cell 145, 941-955 (2011).

9. Nishimura, E. K. et al. Dominant role of the niche in melanocyte stem-cell fate determination. Nature 416, 854 (2002).

10. Nishimura, E. K., Granter, S. R. & Fisher, D. E. Mechanisms of hair graying: incomplete melanocyte stem cell maintenance in the niche. Science 307, 720-724 (2005).

11. Anthony, T. E. et al. Control of stress-induced persistent anxiety by an extra-amygdala septohypothalamic circuit. Cell 156, 522-536 (2014).

12. Ramirez, S. et al. Activating positive memory engrams suppresses depression-like behaviour. Nature 522, 335-339 (2015).

13. Heidt, T. et al. Chronic variable stress activates hematopoietic stem cells. Nat Med 20, 754-758 (2014).

14. Tye, K. M. et al. Dopamine neurons modulate neural encoding and expression of depression-related behaviour. Nature 493, 537-541 (2013).

15. Acs, G., Biro, T., Acs, P., Modarres, S. & Blumberg, P. M. Differential activation and desensitization of sensory neurons by resiniferatoxin. J. Neurosci. 17, 5622-5628 (1997).

16. Baral, P. et al. Nociceptor sensory neurons suppress neutrophil and γδ T cell responses in bacterial lung infections and lethal pneumonia. Nature Medicine 24, 417-426 (2018).

17. Ulrich-Lai, Y. M. & Herman, J. P. Neural regulation of endocrine and autonomic stress responses. Nature Reviews Neuroscience 10, 397-409 (2009).

18. Caterina, M. J. et al. The capsaicin receptor: a heat-activated ion channel in the pain pathway. Nature 389, 816-824 (1997).

19. Kondo, T. & Hearing, V. J. Update on the regulation of mammalian melanocyte function and skin pigmentation. Expert Rev Dermatol 6, 97-108 (2011).

20. Steingrimsson, E., Copeland, N. G. & Jenkins, N. A. Melanocyte stem cell maintenance and hair graying. Cell 121, 9-12 (2005).

21. Liao, C.-P., Booker, R. C., Morrison, S. J. & Le, L. Q. Identification of hair shaft progenitors that create a niche for hair pigmentation. Genes Dev. 31, 744-756 (2017).

22. Inomata, K. et al. Genotoxic stress abrogates renewal of melanocyte stem cells by triggering their differentiation. Cell 137, 1088-1099 (2009).

23. Harris, M. L. et al. A direct link between MITF, innate immunity, and hair graying. PLOS Biology 16, e2003648 (2018).

24. Bosenberg, M. et al. Characterization of melanocyte-specific inducible Cre recombinase transgenic mice. genesis 44, 262-267 (2006).

25. Kohler, C. et al. Mouse cutaneous melanoma induced by mutant BRaf arises from expansion and dedifferentiation of mature pigmented melanocytes. Cell Stem Cell 21, 679-693.e6 (2017).

26. Moon, H. et al. Melanocyte stem cell activation and translocation initiate cutaneous melanoma in response to UV exposure. Cell Stem Cell 21, 665-678.e6 (2017).

27. Sheng, M. & Greenberg, M. E. The regulation and function of c-fos and other immediate early genes in the nervous system. Neuron 4, 477-485 (1990).

28. Kostrzewa, R. M. & Jacobowitz, D. M. Pharmacological actions of 6-hydroxydopamine. Pharmacol Rev 26, 199-288 (1974).

29. Boullin, D. J., Costa, E. & Brodie, B. B. Discharge of tritium-labeled guanethidine by sympathetic nerve stimulation as evidence that guanethidine is a false transmitter. Life Sciences 5, 803-808 (1966).

30. Acar, M. et al. Deep imaging of bone marrow shows non-dividing stem cells are mainly perisinusoidal. Nature 526, 126-130 (2015).

31. Lay, K., Kume, T. & Fuchs, E. FOXC1 maintains the hair follicle stem cell niche and governs stem cell quiescence to preserve long-term tissue-regenerating potential. PNAS 113, E1506-E1515 (2016).

32. Wang, L., Siegenthaler, J. A., Dowell, R. D. & Yi, R. Foxc1 reinforces quiescence in self-renewing hair follicle stem cells. Science 351, 613-617 (2016).

33. Cho, I. J. et al. Mechanisms, hallmarks, and implications of stem cell quiescence. Stem Cell Reports 12, 1190-1200 (2019).

34. Nishimura, E. K. et al. Key roles for transforming growth factor 13 in melanocyte stem cell maintenance. Cell Stem Cell 6, 130-140 (2010).

35. Takeo, M. et al. EdnrB governs regenerative response of melanocyte stem cells by crosstalk with Wnt signaling. Cell Reports 15, 1291-1302 (2016).

36. Tirosh, I. et al. Dissecting the multicellular ecosystem of metastatic melanoma by single-cell RNA-seq. Science 352, 189-196 (2016).

37. Peters, E. M. J., Tobin, D. J., Botchkareva, N., Maurer, M. & Paus, R. Migration of melanoblasts into the developing murine hair follicle is accompanied by transient c-Kit expression. J Histochem Cytochem. 50, 751-766 (2002).

38. Chou, W. C. et al. Direct migration of follicular melanocyte stem cells to the epidermis after wounding or UVB irradiation is dependent on Mc1r signaling. Nature Medicine 19, 924-929 (2013).

39. Losiewicz, M. D., Carlson, B. A., Kaur, G., Sausville, E. A. & Worland, P. J. Potent inhibition of Cdc2 kinase activity by the flavonoid L86-8275. Biochemical and Biophysical Research Communications 201, 589-595 (1994).

40. Wyatt, P. G. et al. Identification of N-(4-Piperidinyl)-4-(2,6-dichlorobenzoylamino)-1H-pyrazole-3-carboxamide (AT7519), a novel cyclin dependent kinase inhibitor using fragment-based X-ray crystallography and structure based drug design. J. Med. Chem. 51, 4986-4999 (2008).

41. Borden, P., Houtz, J., Leach, S. D. & Kuruvilla, R. Sympathetic innervation during development is necessary for pancreatic islet architecture and functional maturation. Cell Rep 4, 287-301 (2013).

42. Zeng, X. et al. Innervation of thermogenic adipose tissue via a calsyntenin 3β-S100b axis. Nature 569, 229 (2019).

43. Katayama, Y. et al. Signals from the sympathetic nervous system regulate hematopoietic stem cell egress from bone marrow. Cell 124, 407-421 (2006).

44. Reed, C. M. The ultrastructure and innervation of muscles controlling chromatophore expansion in the squid, Loligo vulgaris. Cell Tissue Res. 282, 503-512 (1995).

45. Fan, S. M.-Y. et al. External light activates hair follicle stem cells through eyes via an ipRGC-SCN-sympathetic neural pathway. PNAS 115, E6880-E6889 (2018).

46. Lerner, A. B. Gray hair and sympathectomy. Report of a case. Arch Dermatol 93, 235-236 (1966).

47. Ortonne, J. P., Thivolet, J. & Guillet, R. Graying of hair with age and sympathectomy. Arch Dermatol 118, 876-877 (1982).

48. Hinoi, E. et al. The sympathetic tone mediates leptin's inhibition of insulin secretion by modulating osteocalcin bioactivity. J Cell Biol 183, 1235-1242 (2008).

49. Abraira, V. E. et al. The cellular and synaptic architecture of the mechanosensory dorsal horn. Cell 168, 295-310.e19 (2017).

50. Pruitt, S. C., Freeland, A., Rusiniak, M. E., Kunnev, D. & Cady, G. K. Cdknlb overexpression in adult mice alters the balance between genome and tissue ageing. Nat Commun 4, 2626 (2013).

51. Szallasi, A. & Blumberg, P. M. Resiniferatoxin, a phorbol-related diterpene, acts as an ultrapotent analog of capsaicin, the irritant constituent in red pepper. Neuroscience 30, 515-520 (1989).

52. Riol-Blanco, L. et al. Nociceptive sensory neurons drive interleukin-23-mediated psoriasiform skin inflammation. Nature 510, 157-161 (2014).

53. Kashem, S. W. et al. Nociceptive sensory fibers drive interleukin-23 production from CD301b+dermal dendritic cells and drive protective cutaneous immunity. Immunity 43, 515-526 (2015).

54. Marshall, I. C. B. et al. Activation of vanilloid receptor 1 by resiniferatoxin mobilizes calcium from inositol 1,4,5-trisphosphate-sensitive stores. British Journal of Pharmacology 138, 172-176 (2003).

55. Neubert, J. et al. Peripherally induced resiniferatoxin analgesia. Pain 104, 219-228 (2003).

56. Watanabe, T., Sakurada, N. & Kobata, K. Capsaicin-, resiniferatoxin-, and olvanil-induced adrenaline secretions in rats via the vanilloid receptor. Bioscience, Biotechnology, and Biochemistry 65, 2443-2447 (2001).

57. Bing Zhang, He, M. & Hsu, Y.-C. FACS isolation of melanocyte stem cells from mouse skin. Protoc. Exch (2019). DOI: 10.21203/rs.2.17987/v1.

58. Gilchrest, B. A., Vrabel, M. A., Flynn, E. & Szabo, G. Selective cultivation of human melanocytes from newborn and adult epidermis. J. Invest. Dermatol. 83, 370-376 (1984).

59. Dobin, A. et al. STAR: ultrafast universal RNA-seq aligner. Bioinformatics 29, 15-21 (2013).

60. Love, M. I., Huber, W. & Anders, S. Moderated estimation of fold change and dispersion for RNA-seq data with DESeq2. Genome Biology 15, 550 (2014).

61. Huang, D. W., Sherman, B. T. & Lempicki, R. A. Bioinformatics enrichment tools: paths toward the comprehensive functional analysis of large gene lists. Nucleic Acids Res. 37, 1-13 (2009).

62. Huang, D. W., Sherman, B. T. & Lempicki, R. A. Systematic and integrative analysis of large gene lists using DAVID bioinformatics resources. Nat Protoc 4, 44-57 (2009).

1. A method of reducing and/or treating hair greying in a subject comprising inhibiting melanocyte stem cell (MeSC) hyper proliferation.

2. The method of claim 1, wherein MeSC hyperproliferation is inhibited by administering a neurotoxin.

3. The method of claim 1, wherein MeSC hyperproliferation is inhibited by administering 6-hydroxy dopamine or botulinum toxin.

4. The method of claim 1, wherein inhibiting MeSC hyper proliferation comprises inhibiting secretion of norepinephrine from activated sympathetic nerves.

5. The method of claim 4, wherein sympathetic nerves are deactivated.

6. (canceled)

7. The method of claim 4, wherein secretion of norepinephrine is inhibited by administering to the subject an agent selected from the group consisting of: guanethidine, xylocholine, bretylium, debrisoquin, and botulinum toxin.

8. The method of claim 1, wherein inhibition of MeSC hyper proliferation comprises inhibiting an adrenergic receptor.

9. The method of claim 8, wherein the adrenergic receptor is a β2 adrenergic receptor.

10. The method of claim 8, wherein the adrenergic receptor is inhibited by administering to the subject of a beta blocker.

11. (canceled)

12. The method of claim 8, wherein the adrenergic receptor is inhibited by administering to the subject a beta blocker selected from the group consisting of: propranolol, atenolol, metoprolol, acebutolol, nadolol, sotalol, bisoprolol, penbutolol, timolol, betaxolol, labetalol, pindolol, careolol, and exmolol.

13. The method of claim 1, wherein MeSC hyper proliferation is inhibited by administering to the subject a cyclin dependent kinase (CDK) inhibitor or a BRAF inhibitor.

14. The method of claim 13, wherein the CDK inhibitor is selected from the group consisting of: alsterpaullone, aminopurvalanol A, arcyriaflavin A, AZD 5438, BIO, BMS 265246, BRD 6989, BS 181 dihydrochyloride, CGP 60474, CGP 74514 dihydrochloride, (R)-CR8, CVT 313, (R)-DRF053 dihydrochloride, flavopiridol, indirubin-3′-oxime, kenpaullone, LDC 000067, NSC 625987, NSC 663284, NSC 693868, NU 2058, NU 6140, NVP 2, olomoucine, [Ala⁹²]-p16 (84-103), palbociclib, PHA 767491 hydrochloride, purvalanol A, purvalanol B, R 547, ribociclib, Ro3306, roscovitine, ryuvidine, senexin A, SNS 032, SU 9516, letrozole, fulvestrant, dinaciclib, and AT7519.

15. (canceled)

16. The method of claim 13, wherein the BRAF inhibitor is vemurafenib or dabrafenib.

17. The method of claim 1, wherein MeSC hyperproliferation is inhibited by inhibiting release of norepinephrine from sympathetic nerves and by blocking one or more adrenergic receptors.

18. The method of claim 1, wherein MeSC hyper proliferation is inhibited by administering to the subject a CDK inhibitor and/or a beta blocker.

19. (canceled)

20. The method of claim 18, wherein the CDK inhibitor and/or the beta blocker is administered to the subject topically.

21. The method of claim 18, wherein the CDK inhibitor and/or the beta blocker is administered to the subject orally.

22.-46. (canceled)

47. A pharmaceutical composition comprising a beta blocker and a CDK inhibitor, wherein the pharmaceutical composition is formulated for topical administration to a subject exhibiting hair greying.

48. A method of causing or accelerating hair greying in a subject comprising increasing levels of norepinephrine in the subject by administering an agent.

49. The method of claim 48, wherein the agent is selected from the group consisting of norepinephrine, an Akt activator and an adrenergic beta agonist.

50.-52 (canceled)
